SEQUENCE LISTING (76 sequences)

SEQ ID NO: 1 - DNA, 1416 bp, Bacillus thuringiensis
  source 1..1416: mol_type = genomic DNA
atggataaatttattacagacttaatacaagataaatcaatagtacgacaacttcaaattttagaaaccttgattgatagcaatgaaataaagtcttccaaggatttatcacaaagtttaaagtgtacaagtagaacaataataaatgacatttctcagttaaaactagcgcttcccgaaaattgggatttaataagtgttcaatccaaagggtacctattaaaacgtgatttttcaaacgatttctccgaattaattattccctatttaatgaatagtgaactttatacaatattaatcggtatatttaatcaaaaatattattctttagaaaaatggtcccagttgttatatctagataaaattacactaaaaaaaatgctgaaaaactttcggaagatacttgtgaactttggattggattttaattttagaacgattaaattgattggccaagaaataaacttaagatatttttatataatgttcttttataatatccaaaagtataaagaagtaatcaatttagattctagattacaagaaaaaattaaaagcattactagaattcacaatgtagaaatagattataatatgctagcagttattattagtgtatctataaagagaattgcaaataaacattatatgtcagaagttttagagtttcttcctatacttgatacaaataatttaaattgcataagttcaattatatgcgaattggaaattttcttcaatataaaattcacagaatatgaattaagttattttaagaatgctttttcaataatattagaatggaacattgaagaaaaaaataggatcacagattattattataaaataaacaaaaagacttatgataaaaacaaacatttattccaaacaatatcttctgaaataaatgtttgtttagaaataaaagaaaagataaaacatgacttgtatttcattttacataaaatttacaagtttcaaaagtatggtttatcaataggcgcttttggtaatgaatttgacactgtacatcatgaattttcagaaggacataatagaatttatcctcttatttcttcttggaataaaaaaataaataaaagtagattaactaacgatgagatcaattacataatataccatattttatttattgtccattcaaatcataataaaaaaggattgttattattatctggttcatcagctttaaagaagtttatttattataaactcaatcacgagctaggtgattttgtaacactacaacaaaaaccagattgtatgcataaatttgatgttattctgacaaattatcaaatcccgaataccccaatacctataattcggatctcaaacaaaacaattcaaaaagattcaagttatgttagaaaagttttatccccttttaactaa

SEQ ID NO: 2 - protein, 471 aa, Bacillus thuringiensis
MDKFITDLIQDKSIVRQLQILETLIDSNEIKSSKDLSQSLKCTSRTIINDISQLKLALPENWDLISVQSKGYLLKRDFSNDFSELIIPYLMNSELYTILIGIFNQKYYSLEKWSQLLYLDKITLKKMLKNFRKILVNFGLDFNFRTIKLIGQEINLRYFYIMFFYNIQKYKEVINLDSRLQEKIKSITRIHNVEIDYNMLAVIISVSIKRIANKHYMSEVLEFLPILDTNNLNCISSIICELEIFFNIKFTEYELSYFKNAFSIILEWNIEEKNRITDYYYKINKKTYDKNKHLFQTISSEINVCLEIKEKIKHDLYFILHKIYKFQKYGLSIGAFGNEFDTVHHEFSEGHNRIYPLISSWNKKINKSRLTNDEINYIIYHILFIVHSNHNKKGLLLLSGSSALKKFIYYKLNHELGDFVTLQQKPDCMHKFDVILTNYQIPNTPIPIIRISNKTIQKDSSYVRKVLSPFN

SEQ ID NO: 3 - DNA, 717 bp, Bacillus thuringiensis
  source 1..717: mol_type = genomic DNA
gactgtccttcttatcttacacgctataatgcggatattaactcaataagccataaggcttctaatcgcaaggactgctggggtagttcaatagctctggtataaatagcagtaaaataacctttggtacaattcgatagttactagacgtaacgattagccgagaaatccccactttagagagcacgttaggaggttaagtgaagggtatttcacaaatgatcgattatatttataggaaaaggatagatcacttcatctatagatgaaattatctatccttttatttgttagataggagatataatttactcatagccccagagaaagtgttaacaggtgatagcatccaattttatagtaagggaggttttactaagttctaataattttctgggtaattagtccttacaacgacttacatttaggaaattgcctgttaataaaatttatatagtaaaggagtgctttaatgtaaaaaaatagggataaatgaatttttgtgaataggaagaccatttgataggtaaatattataattcaaaaagtagaataagcaaaattgagtaacacattaatgatataaactaattttatacaattgaaattgataaaaagttatgagtgtttaataatcagtaattaccaataaagaattaagaatacaagtttacaagaaataagtgttacaaaaaatagctgaaaaggaagatgaac

SEQ ID NO: 4 - DNA, 30 bp, Bacillus thuringiensis
  misc_feature 4..7: note = N may be A, T, C or G
  misc_feature 10: note = N may be A, T, C or G
  misc_feature 12..13: note = N may be A, T, C or G
  misc_feature 17..18: note = N may be A, T, C or G
  misc_feature 22: note = N may be A, T, C or G
  misc_feature 24: note = N may be A, T, C or G
  misc_feature 26: note = N may be A, T, C or G
  source 1..30: mol_type = other DNA
ttcnnnnatngnngaanntatntntncttt

SEQ ID NO: 5 - DNA, 1226 bp, Bacillus thuringiensis
  source 1..1226: mol_type = genomic DNA
ttttgtacatgcttaaacaagcgaaaatgtacatgtttatcttgacatttacaaagagatgaatatatttgaattttattaattaaatccagaaaaaaatatcagaatagaaaatgctttaaggcctatttgcagttgaatatttatggtatgaatattgccgtatttcttagtatgctaaaaatacaagcctgtaaatcactttgttatgactgtaaagtaaaagaaggatattgaactattttattatttataaggtattatatatactttgagattcagaaggtttgaataagttacgctttatatgccgatattttggttcaactagagtatcaaatgtggaatatttctttgctatataaaacctttacttcttaatcttatataagcacaaagtaaaacataaggggatttttgaaaaaagagtattatggtctagtataaaacttgtattttagaactttaaatatattaagataattcaaaggggaaaataaaatccaataatcattaagatggtagtttctttggattcgtttcttaaaatatctatttttgattttccttatctattttgtaataaaacttttcattaatagatgaaaatatgtatgctttattaggaaataattaaggtatcttttaattatctataattatttgttatagaggaattagataattgaagttcttttcatttgggaatacggtgataaaaaaggagggacggagttataaggatcaataagaatacataattaaattgttttacaataaaacattccattctcattaatattcttataatcacaaaaaccaaatgctataaaatggattgtttgaaaaagttgtgaatatgcatatttttggatgcgtgtacaattataatcatcatgatatcacttctcttttaagaatgcgaagttttgtcgttatgtaaacatgggtgctcaaaactttacattttttgtacaatataaaatattaaaatacagttgtacagataattgatatttctttatatgaaaaaatgcttataaatactatgcaaagctaacgtataattttttagaccttatagaatcaaataagaataaagatagaaaataaaattgaaatactaaggctagcaaagtaacgtgaggtaatagcatctattctgagttttgtagttaaatgttacctagtgataagtaattcagtattaagggaggagaaatgta

SEQ ID NO: 6 - DNA, 115 bp, Bacillus thuringiensis
  source 1..115: mol_type = genomic DNA
cctaaaatttcaaggcattttaaatgttttgtctagaaaagtatggataacttcatgaatgggtgaaaatatctatgcttttttattacaagacaaagatatactgtactcataa

SEQ ID NO: 7 - DNA, 115 bp, Bacillus thuringiensis
  source 1..115: mol_type = genomic DNA
gcttttaagaacccctattttaaatgttttgtctagaaaagtatggataacttcatgaatgggtgaaaatatctatgcttttttattacaagacaaagatatactgtactcataa

SEQ ID NO: 8 - DNA, 115 bp, Bacillus thuringiensis
  source 1..115: mol_type = genomic DNA
tattttctagtaatacatatgtatagagcaacttaatcaagcagagatattttcacctatcgatgaaaatatctctgctttttctttttttatttggtatatgctttacttgtaa

SEQ ID NO: 9 - DNA, 115 bp, Bacillus thuringiensis
  source 1..115: mol_type = genomic DNA
cacttctaaatgaagtgaaagtgggggtagttcaaaaaaagcatagatatcttcttctataggtgaagatatctatgctttttctttttaaattaaagatatactttactcatac

SEQ ID NO: 10 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 11 - protein, 719 aa, Bacillus thuringiensis
MKLKNQDKHQSFSSNAKVDKISTDSLKNETDIELQNINHEDCLKMSEYENVEPFVSASTIQTGIGIAGKILGTLGVPFAGQVASLYSFILGELWPKGKNQWEIFMEHVEEIINQKISTYARNKALTDLKGLGDALAVYHDSLESWVGNRNNTRARSVVKSQYIALELMFVQKLPSFAVSGEEVPLLPIYAQAANLHLLLLRDASIFGKEWGLSSSEISTFYNRQVERAGDYSDHCVKWYSTGLNNLRGTNAESWVRYNQFRRDMTLMVLDLVALFPSYDTQMYPIKTTAQLTREVYTDAIGTVHPHPSFTSTTWYNNNAPSFSAIEAAVVRNPHLLDFLEQVTIYSLLSRWSNTQYMNMWGGHKLEFRTIGGTLNISTQGSTNTSINPVTLPFTSRDVYRTESLAGLNLFLTQPVNGVPRVDFHWKFVTHPIASDNFYYPGYAGIGTQLQDSENELPPEATGQPNYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTVGFTTPFSFLDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYAKQLHIERNM

SEQ ID NO: 12 - protein, 633 aa, Bacillus thuringiensis
MNSVLNSGRTTICDAYNVAAHDPFSFQHKSLDTVQKEWTEWKKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQFQIQGYQLLLLPLFAQAANLHLSFIRDVILNADEWGISAATLRTYRDYLKNYTRDYSNYCINTYQSAFKGLNTRLHDMLEFRTYMFLNVFEYVSIWSLFKYQSLLVSSGANLYASGSGPQQTQSFTSQDWPFLYSLFQVNSNYVLNGFSGARLSNTFPNIVGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTLNSGTQFDLMNIMLVPTNISPLY

SEQ ID NO: 13 - DNA, 15 bp, Bacillus thuringiensis
  source 1..15: mol_type = genomic DNA
gaaaggagggatgcc

SEQ ID NO: 14 - DNA, 180 bp, Bacillus thuringiensis
  source 1..180: mol_type = genomic DNA
aaactcaggtttaaatatcgttttcaaatcaattgtccaagagcagcattacaaatagataagtaatttgttgtaatgaaaaacggacatcacctccattgaaacggagtgatgtccgttttactatgttattttctagtaatacatatgtatagagcaacttaatcaagcagagatatt

SEQ ID NO: 15 - DNA, 115 bp, Bacillus thuringiensis
  source 1..115: mol_type = genomic DNA
aagggtatttcacaaatgatcgattatatttataggaaaaggatagatcacttcatctatagatgaaattatctatccttttatttgttagataggagatataatttactcatag

SEQ ID NO: 16 - DNA, 32 bp, Bacillus thuringiensis
  source 1..32: mol_type = genomic DNA
cccaagcttgactgtccttcttatcttacacg

SEQ ID NO: 17 - DNA, 31 bp, Bacillus thuringiensis
  source 1..31: mol_type = genomic DNA
cgggatccttttcagctattttttgtaacac

SEQ ID NO: 18 - DNA, 25 bp, Bacillus thuringiensis
  source 1..25: mol_type = genomic DNA
acatcctccctacactttctaatac

SEQ ID NO: 19 - DNA, 23 bp, Bacillus thuringiensis
  source 1..23: mol_type = genomic DNA
tcttctatggacccgttctctac

SEQ ID NO: 20 - DNA, 16 bp, Bacillus thuringiensis
  source 1..16: mol_type = genomic DNA
cagtggcaaatccata

SEQ ID NO: 21 - DNA, 21 bp, Bacillus thuringiensis
  source 1..21: mol_type = genomic DNA
cttggtaaggctcttgtgctt

SEQ ID NO: 22 - DNA, 28 bp, Bacillus thuringiensis
  source 1..28: mol_type = genomic DNA
gttcatgttcatcttccttttcagctat

SEQ ID NO: 23 - DNA, 17 bp, Bacillus thuringiensis
  source 1..17: mol_type = genomic DNA
tgctatcaatcaaggtt

SEQ ID NO: 24 - DNA, 25 bp, Bacillus thuringiensis
  source 1..25: mol_type = genomic DNA
gttgtcgtactattgatttatcttg

SEQ ID NO: 25 - DNA, 20 bp, Bacillus thuringiensis
  source 1..20: mol_type = genomic DNA
ccattacatttctcctccct

SEQ ID NO: 26 - DNA, 23 bp, Bacillus thuringiensis
  source 1..23: mol_type = genomic DNA
cgaatccaaagaaactaccatct

SEQ ID NO: 27 - DNA, 31 bp, Bacillus thuringiensis
  source 1..31: mol_type = genomic DNA
cccaagcttttttgtacatgcttaaacaagc

SEQ ID NO: 28 - DNA, 26 bp, Bacillus thuringiensis
  source 1..26: mol_type = genomic DNA
cccaagcttacttaaggttttagttc

SEQ ID NO: 29 - DNA, 27 bp, Bacillus thuringiensis
  source 1..27: mol_type = genomic DNA
cccaagcttcgcttcccgaaaattggg

SEQ ID NO: 30 - DNA, 42 bp, Bacillus thuringiensis
  source 1..42: mol_type = genomic DNA
tattattctttagaaaaagcggcccagttgttatatctagat

SEQ ID NO: 31 - DNA, 42 bp, Bacillus thuringiensis
  source 1..42: mol_type = genomic DNA
atctagatataacaactgggccgctttttctaaagaataata

SEQ ID NO: 32 - DNA, 29 bp, Bacillus thuringiensis
  source 1..29: mol_type = genomic DNA
ggggatccgttttgtagttaaatgttacc

SEQ ID NO: 33 - DNA, 35 bp, Bacillus thuringiensis
  source 1..35: mol_type = genomic DNA
cgggatcccgggttagttaaaaggggataaaactt

SEQ ID NO: 34 - DNA, 26 bp, Bacillus thuringiensis
  source 1..26: mol_type = genomic DNA
cgggatccgatataacaactgggacc

SEQ ID NO: 35 - DNA, 30 bp, Bacillus thuringiensis
  source 1..30: mol_type = genomic DNA
cgggatccttagttaaaaggggataaaact

SEQ ID NO: 36 - DNA, 32 bp, Bacillus thuringiensis
  source 1..32: mol_type = genomic DNA
aaaactgcagttttgtacatgcttaaacaagc

SEQ ID NO: 37 - DNA, 31 bp, Bacillus thuringiensis
  source 1..31: mol_type = genomic DNA
cccaagcttttagttaaaaggggataaaact

SEQ ID NO: 38 - DNA, 33 bp, Bacillus thuringiensis
  source 1..33: mol_type = genomic DNA
aaaactgcaggactgtccttcttatcttacacg

SEQ ID NO: 39 - DNA, 37 bp, Bacillus thuringiensis
  source 1..37: mol_type = genomic DNA
ggaattccatatgaacaagaataatactaaattaagc

SEQ ID NO: 40 - DNA, 37 bp, Bacillus thuringiensis
  source 1..37: mol_type = genomic DNA
cgggatcccgttacttaatagagacatcgtaaaaatg

SEQ ID NO: 41 - DNA, 26 bp, Bacillus thuringiensis
  source 1..26: mol_type = genomic DNA
gcataagttcaattatatgcgaattg

SEQ ID NO: 42 - DNA, 29 bp, Bacillus thuringiensis
  source 1..29: mol_type = genomic DNA
tttcagaagatattgtttggaataaatgt

SEQ ID NO: 43 - protein, 33 aa, Bacillus thuringiensis
EGCQAESICKEFYISSSSLYRIISQINKVIKRQ

SEQ ID NO: 44 - DNA, 42 bp, Bacillus thuringiensis
  source 1..42: mol_type = genomic DNA
tattattctttagaaaaatggtcccagttgttatatctagat

SEQ ID NO: 45 - protein, 14 aa, Bacillus thuringiensis
YYSLEKWSQLLYLD

SEQ ID NO: 46 - DNA, 42 bp, Bacillus thuringiensis; identical to SEQ ID NO: 30
  source 1..42: mol_type = genomic DNA

SEQ ID NO: 47 - protein, 14 aa, Bacillus thuringiensis
YYSLEKAAQLLYLD

SEQ ID NO: 48 - DNA, 52 bp, Bacillus thuringiensis
  source 1..52: mol_type = genomic DNA
aggaaaaccatacatcacttcatctatagaacaaattatctatccttttatt

SEQ ID NO: 49 - DNA, 31 bp, Bacillus thuringiensis
  source 1..31: mol_type = genomic DNA
cttcctctatagaggaagatatctatgcttt

SEQ ID NO: 50 - DNA, 96 bp, Bacillus thuringiensis
  source 1..96: mol_type = genomic DNA
ttataggaaaaggatagatcacttcatctatagatgaaattatctatccttttatttgttagataggagatataatttactcatagccccagagaa

SEQ ID NO: 51 - protein, 32 aa, Bacillus thuringiensis
KYYSLEKWSQLLYLDKITLKKMLKNFRKILVN

SEQ ID NO: 52 - DNA, 52 bp, Bacillus thuringiensis
  source 1..52: mol_type = genomic DNA
aggaaaaggatagatcacttcatctatagatgaaattatctatccttttatt

SEQ ID NO: 53 - DNA, 194 bp, Bacillus thuringiensis
  source 1..194: mol_type = genomic DNA
aaaatacaagcctgtaaatcactttgttatgactgtaaagtaaaagaaggatattgaactattttattatttataaggtattatatatactttgagaatctattttgtaataaaacttttcattaatagatgaaaatatgtatgctttattaggaaataattaaggtatcttttaattatctataattatttgt

SEQ ID NO: 54 - DNA, 36 bp, Bacillus thuringiensis
  source 1..36: mol_type = genomic DNA
gatagatcacttcatctatagatgaaattatctatc

SEQ ID NO: 55 - DNA, 36 bp, Bacillus thuringiensis
  source 1..36: mol_type = genomic DNA
aataaaacttttcattaatagatgaaaatatgtatg

SEQ ID NO: 56 - DNA, 32 bp, Bacillus thuringiensis
  source 1..32: mol_type = genomic DNA
tttcattaatagatgaaaatatgtatgcttta

SEQ ID NO: 57 - DNA, 32 bp, Bacillus thuringiensis
  source 1..32: mol_type = genomic DNA
cttcatctatagatgaaattatctatcctttt

SEQ ID NO: 58 - DNA, 32 bp, Bacillus thuringiensis
  source 1..32: mol_type = genomic DNA
cttcttctataggtgaagatatctatgctttt

SEQ ID NO: 59 - DNA, 32 bp, Bacillus thuringiensis
  source 1..32: mol_type = genomic DNA
cttcatgaatgggtgaaaatatctatgctttt

SEQ ID NO: 60 - DNA, 32 bp, Bacillus thuringiensis
  source 1..32: mol_type = genomic DNA
tttcacctatcgatgaaaatatctctgctttt

SEQ ID NO: 61 - DNA, 32 bp, Bacillus thuringiensis; identical to SEQ ID NO: 59
  source 1..32: mol_type = genomic DNA

SEQ ID NO: 62 - DNA, 32 bp, Bacillus thuringiensis; identical to SEQ ID NO: 59
  source 1..32: mol_type = genomic DNA

SEQ ID NO: 63 - DNA, 32 bp, Bacillus thuringiensis
  source 1..32: mol_type = genomic DNA
cttcatcaataggtgaaaatatctatgctttt

SEQ ID NO: 64 - DNA, 32 bp, Bacillus thuringiensis; identical to SEQ ID NO: 59
  source 1..32: mol_type = genomic DNA

SEQ ID NO: 65 - DNA, 199 bp, Bacillus thuringiensis
  source 1..199: mol_type = genomic DNA
atcacgaaaagaagtttttaagaaccattcctaaaatttcaaggcattttagatgttttatgtggaaaagcatggttaacttcatcaataggtgaaaatatctatgcttttttatttcaggacagagatatactgtactcataaatatagaaatatgtatttaagaaagattgaaagcgtattaaataatctgatagga

SEQ ID NO: 66 - DNA, 343 bp, Bacillus thuringiensis
  source 1..343: mol_type = genomic DNA
tgtagaaactttgtaatttgtatgaattgagggattgaattccttgtgttaattaacatatatatcgatttgatataaagataggatctaaaaggagctaacaaactgttagctccttttagatcctttttaagtatatcgcacaaaaatcaaatcatacatcacgaaaagtagcttttaagaacccctattttaaatgttttgtctagaaaagtatggataacttcatgaatgggtgaaaatatctatgcttttttattacaagacaaagatatactgtactcataaatgtagaaatatctatttaagaaagattgaaatcatatccaataatctgatagga

SEQ ID NO: 67 - DNA, 40 bp, Bacillus thuringiensis
  source 1..40: mol_type = genomic DNA
cccaagcttgtgatgtccgttttactatgttattttctag

SEQ ID NO: 68 - DNA, 40 bp, Bacillus thuringiensis
  source 1..40: mol_type = genomic DNA
cgggatccacttatactattatttaattttcagatataat

SEQ ID NO: 69 - DNA, 40 bp, Bacillus thuringiensis
  source 1..40: mol_type = genomic DNA
cccaagcttatcacgaaaagaagtttttaagaaccattcc

SEQ ID NO: 70 - DNA, 40 bp, Bacillus thuringiensis
  source 1..40: mol_type = genomic DNA
cgggatcctatcagattattggatatgatttcaatctttc

SEQ ID NO: 71 - DNA, 40 bp, Bacillus thuringiensis
  source 1..40: mol_type = genomic DNA
cccaagctttgtagaaactttgtaatttgtatgaattgag

SEQ ID NO: 72 - DNA, 115 bp, Bacillus thuringiensis; identical to SEQ ID NO: 7
  source 1..115: mol_type = genomic DNA

SEQ ID NO: 73 - DNA, 772 bp, Bacillus thuringiensis
  source 1..772: mol_type = genomic DNA
acttaggacattgaactactcaccacttaacgtccttacagactttttgaagtgggagattcctgctaagataaccaatgtatccaccggttatcttagcaggcgctacccgtagtccctacagtgagaacataagtatcttgtacattgacgcaccacactccctactttagtttggttttcgcaacttcggcaagcatggggcgaaagaacgttgaagattttacagttgcaacgtttttctgaaaccaacctcataacttcagttttcgaagagcaatctgtataagtaaatgatagcaaatatgttggcttatgcctatcgatattcatatcccacctaaaattggtgtttcacaccttgacatttttgaggaaggagtcttctgttggaaaacgataaaatctttttaattacttgaattctaagtgtagaaactttgtaatttgtatgaattgagggattgaattccttgtgttaattaacatatatatcgatttgatataaagataggatctaaaaggagctaacaaactgttagctccttttagatcctttttaagtatatcgcacaaaaatcaaatcatacatcacgaaaagtagcttttaagaacccctattttaaatgttttgtctagaaaagtatggataacttcatgaatgggtgaaaatatctatgcttttttattacaagacaaagatatactgtactcataaatgtagaaatatctatttaagaaagattgaaatcatatccaataatctgatagg

SEQ ID NO: 74 - DNA, 115 bp, Bacillus thuringiensis
  source 1..115: mol_type = genomic DNA
cctaaaatttcaaggcattttagatgttttatgtggaaaagcatggttaacttcatcaataggtgaaaatatctatgcttttttatttcaggacagagatatactgtactcataa

SEQ ID NO: 75 - DNA, 115 bp, Bacillus thuringiensis; identical to SEQ ID NO: 7
  source 1..115: mol_type = genomic DNA

SEQ ID NO: 76 - DNA, 381 bp, Bacillus thuringiensis
  source 1..381: mol_type = genomic DNA
gtgatgtccgttttactatgttattttctagtaatacatatgtatagagcaacttaatcaagcagagatattttcacctatcgatgaaaatatctctgctttttctttttttatttggtatatgctttacttgtaatcgaaaataaagcactaataagagtatttataggtgtttgaagttatttcagttcatttttaaagaaggtttaaagacgttagaaagttattaaggaataatatttattagtaaattccacatatattatataattaattatgaaatatatgtataaattgaaaatgctttatttgacattacagctaagtataattttgtatgaataaaattatatctgaaaattaaataatagtataagtgga

(19) United States
(12) Patent Application Publication
(10) Pub. No.: US 2025/0228250 A1
(43) Pub. Date: Jul. 17, 2025

(54) NOVEL REGULATOR VIPR IN BACILLUS STRAINS

(71) Applicants: INSTITUT NATIONAL DE RECHERCHE POUR L’AGRICULTURE, L’ALIMENTATION ET L’ENVIRONNEMENT, Paris (FR); INSTITUT NATIONAL DES SCIENCES ET INDUSTRIES DU VIVANT ET DE L’ENVIRONNEMENT, Palaiseau (FR); UNIVERSITE PARIS-SACLAY, GIF-SUR-YVETTE (FR)

(72) Inventors: Haibo Chen, Hunan (CN); Emilie Verplaetse, SACLAY (FR); Didier Lereclus, Versailles (FR); Leyla Slamti, Paris (FR)

(73) Assignees: INSTITUT NATIONAL DE RECHERCHE POUR L’AGRICULTURE, L’ALIMENTATION ET L’ENVIRONNEMENT, Paris (FR); INSTITUT NATIONAL DES SCIENCES ET INDUSTRIES DU VIVANT ET DE L’ENVIRONNEMENT, Palaiseau (FR); UNIVERSITE PARIS-SACLAY, GIF-SUR-YVETTE (FR)

(21) Appl. No.: 18/854,190

(22) PCT Filed: Mar. 31, 2023
(86) PCT No.: PCT/EP2023/058561

§ 371 Date: Oct. 4, 2024

(30) Foreign Application Priority Data
Apr. 4, 2022 (EP) 22305443.8

(51) Int. Cl. A01N 63/23; A01P 7/04; C12N 9/22; C12N 15/75; C12P 21/00

(52) U.S. Cl. CPC A01N 63/23; A01P 7/04; C12N 9/22; C12N 15/75; C12P 21/00; C12N 2800/101; C12N 2830/002

(57) ABSTRACT

The present invention relates to a novel regulator VipR able to regulate the expression of genes, and more specifically the expression of vip3A. It also relates to an expression system allowing the expression of a protein of interest through the VipR regulation. This expression system can be used to transform Bacillus strains.

The present invention relates to a novel regulator VipR able to regulate the expression of genes, and more specifically the expression of vip3A. It also relates to an expression system allowing the expression of a protein of interest through the VipR regulation. This expression system can be used to transform Bacillus strains.

The Bacillus thuringiensis species includes a large number of Gram-positive spore-forming bacterial strains belonging to the Bacillus cereus group and distinguished by the production of parasporal crystal inclusions (1). Many strains of B. thuringiensis are entomopathogenic and their insecticidal properties are primarily due to the crystal proteins that consist of Cry and Cyt toxins, encoded by plasmid genes (2-4). The presence of cry genes is the common feature of all strains of the B. thuringiensis species, and the expression of most cry genes is dependent on the sporulation-specific factors Sigma E and Sigma K, which are functional in the mother cell compartment during sporulation. However, a few cry genes do not follow the same regulation pathway (5). They are also expressed in the mother cell or in a non-sporulating subpopulation during the sporulation process, but independently of the sporulation sigma factors. These are notably the cry3 genes encoding toxins active against coleopteran insects (6) and the cry genes of strain LM1212 which are expressed under the control of a specific transcriptional activator, CpcR, encoded by a plasmid gene (7). Different combinations of toxins can be found in the crystal inclusions depending on the B. thuringiensis strains. As an example, the commercial strain kurstaki HD1 contains 6 cry genes: cry1Aa, cry1Ab, cry1Ac, cry1Ia, cry2Aa and cry2Ab (http://www.lifesci.sussex.ac.uk/home/Neil_Crickmore/Bt/). All these cry genes encode proteins active against lepidopteran larvae and confer a broad insecticidal spectrum within this insect order (8). However, the pathogenicity of B. thuringiensis towards some insects and other arthropods also depends on various chromosomal factors including those belonging to the PIcR virulence regulon (9, 10). Moreover, several B. thuringiensis strains harbor plasmid genes encoding insecticidal proteins other than the Cry toxins. This is notably the case of the Vegetative insecticidal protein Vip3A toxins which contribute significantly to the overall insecticidal activity of the B. thuringiensis strains (11). This type of insecticidal toxin was first discovered in the culture supernatant of the B. thuringiensis strain AB88 and its production was detected from the vegetative growth phase to the end of the stationary phase and sporulation (12).

It has been shown that the vip3A gene is present in about 50% of the B. thuringiensis strains tested and is located on large plasmids also harboring cry1I and cry2A genes (13, 14). Complete sequencing of the B. thuringiensis strain kurstaki HD1 indicates that the vip3A gene is located on the large plasmid pBMB299 which also carries the cry1Aa, cry1Ia, cry2Aa and cry2Ab genes (15). The Vip3A toxins are specifically toxic against lepidopteran insects belonging to the Noctuidae family, including important agricultural pests like Spodoptera exigua and Spodoptera frugiperda which are poorly susceptible to the Cry1A and Cry2A toxins (16, 17). Moreover, the specific receptors recognized by Vip3A toxins in the insect midgut are different from those recognized by the Cry toxins (18-20). These properties mean that vip3A genes are very often used in combination with cry genes in plant transgenesis pyramid strategies to increase plant resistance to insect pests but also to bypass the resistance acquired by certain insects to Cry toxins (21).

Surprisingly, the Inventors have discovered that the upstream region of vip3A is not sufficient to induce the expression of gene fused with this region in the strain B. thuringiensis strain kurstaki HD73 Cry−. Then, the Inventors have shown that the expression of Vip3A in the HD73 strain containing pBMB299 is controlled by a regulator whose gene is located on this same plasmid. This regulator gene called vipR is responsible for vip3A gene transcription during the stationary phase. They also demonstrated that vipR transcription is positively autoregulated and the determination of the vipR and vip3A promoters pinpointed a putative VipR target upstream from the Sigma A-specific −10 region of the vip3A and vipR promoters. Surprisingly, this conserved sequence was also found upstream of cry1I and cry2 genes. Finally, they showed that vip3A and vipR expression is drastically increased in a Δspo0A mutant unable to initiate sporulation. In conclusion, a novel regulator involved in the entomopathogenic potency of B. thuringiensis through a sporulation-independent pathway has been characterized.

The Inventors have then developed an expression system comprising the regulator VipR and its promoter PvipR, a promoter regulated by VipR and a gene coding for a protein of interest. In such expression system, the promoter regulated by VipR allows the transcription of the gene encoding for the protein of interest. This expression system may be used to transform Bacillus strains allowing them to produce proteins of interest.

The present invention relates to the use of the vipR regulator gene in a Bacillus strain having at least 90%, preferably 95%, and more preferably at least 98% or 100% identity with the sequence SEQ ID No1 for directing the expression of at least one promoter comprising the sequence:

5′ TTC-N-N-N-N-AT-N-G-N-N-GAA-N-N-TAT-N-T-N-T-N-CTTT 3′ (SEQ ID No4),
wherein N is A or T or C or G, and
wherein said sequence of SEQ ID No4 is positioned upstream between 15 and 21 nucleotides, more preferably 18 nucleotides, of the Sigma A-specific −10 box preceding the start codon of the gene encoding a protein of interest.

According to a specific embodiment, the vipR regulator gene is used together with the promoter PvipR (SEQ ID No5) which directs the expression of said VipR regulator. In such embodiment, the vipR regulator gene and its promoter PvipR (SEQ ID No5) may be combined in an expression system. The Sigma A-specific −10 box has the following sequence: TNNNNT with N is C or A or G or T, preferably TATAAT or TATACT or TATGCT or TATCGT or TATCTT or TATATT or TATTAT or TATAGT or TATGAT and more preferably TATAAT or TATACT.

Preferably the SEQ ID No4 is the sequence: 5′ TTC-X1-X2-X3-X4-AT-X5-G-X6-X7-GAA-X9-X4-TAT-X8-T-X9-T-X8-CTTT 3′ or more preferably 5′ TTC-X1-X2-X3-X4-AT-X5-G-X6-X7-GAA-X6-X4-TAT-X8-T-X9-T-X8-CTTT 3′, wherein X1 is A or T or C; X2 is C or T; X3 is C or G or T; X4 is A or T; X5 is A or C or G; X6 is A or G; X7 is T or G; X8 is G or C; X9 is A or C; in a preferred embodiment, the sequence is SEQ ID No4: 5′ X1-TTC-X2-X1-X3-X4-AT-X5-G-X6-X7-GAA-X9-X4-TAT-X8-T-X9-T-X8-CTTT-X4 3′ or more preferably 5′ X2-TTC-X1-X2-X3-X4-AT-X5-G-X6-X7-GAA-X6-X4-TAT-X8-T-X9-T-X8-CTTT-X4 3′.

The transcriptional VipR regulator has at least 90%, preferably 95%, and more preferably at least 98% or 100% identity with the sequence SEQ ID No2.

The promoters regulated by transcriptional VipR regulator may be selected in the group consisting of PvipR (SEQ ID No5), Pvip3 (SEQ ID No15 or SEQ ID No3), Pamidase-1 (SEQ ID No6), Pamidase-2 (SEQ ID No7), Pcry1I (SEQ ID No8 or SEQ ID No76), Pcry2Ab (SEQ ID No9) Pamidase_{pBMB65} (SEQ ID No72 or SEQ ID No73), Pamidase_{pBMB95} (SEQ ID No65 or SEQ ID No74) and Pamidase_{pHT73} (SEQ ID No66 or SEQ ID No75), preferably the promoters are selected in the group consisting of PvipR, Pvip3 and Pcry1I and more preferably the promoter is Pvip3.

The term “protein of interest” means an endogenous protein or a protein which is not naturally expressed by the bacterial strain according to the invention, also referred to as a heterologous protein. Preferably, the protein of interest is a cytotoxic protein naturally expressed by a strain of the Bacillus genus or a protein of industrial interest such as enzymes, such as proteases, lipases, amylases; hormones; antigens, for example, usable as immunogens, peptides or proteins for therapeutic use; the protein of interest can thus find application in the field of crop protection, vector control, the commercial production of enzymes and the pharmaceutical industry, in particular for the production of vaccines.

In a particular embodiment, the transcriptional VipR regulator activates expression of genes encoding proteins of interest selected in the group consisting of Vip3A (SEQ ID No10), Cry1I (SEQ ID No11) and Cry2Ab (SEQ ID No12), preferably the protein of interest is selected in the group consisting of Vip3A and Cry1I and more preferably the protein of interest is Vip3A.

The transcriptional VipR regulator activates the expression of genes during the stationary phase, continues for several hours at a high level when bacteria are grown in a rich medium such as LB and preferably the activation occurs at the onset of the stationary phase.

Preferably the Bacillus strain is chosen among Bacillus thuringiensis, Bacillus cereus, Bacillus weihenstephanensis, Bacillus subtilis, Bacillus megaterium, Bacillus brevis, and more preferably Bacillus thuringiensis.

The present invention thus also relates to an expression system of a protein of interest. This expression system is understood as a system comprising several components (sequences) allowing the activation of the expression of a protein of interest by a regulator.

The present invention further relates to an expression system of a protein of interest in a Bacillus strain comprising:
at least one polynucleotide sequence encoding for said protein of interest;
at least one first promoter comprising a sequence:

(SEQ ID Nº 4)
5′ TTC-N-N-N-N-AT-N-G-N-N-GAA-N-
N-TAT-N-T-N-T-N-CTTT 3′,

wherein N is A or T or C or G, and
wherein said sequence of SEQ ID No4 is positioned upstream between 15 and 21 nucleotides of the Sigma A-specific −10 box preceding the start codon of the gene encoding a protein of interest and more preferably 18 nucleotides;
allowing the expression of said polynucleotide sequence encoding for said protein of interest; and
the vipR regulator gene having at least 90%, preferably 95%, and more preferably at least 98% or 100% identity with SEQ ID No1 which directs the expression of said at least one first promoter, and a second promoter PvipR of SEQ ID No5 which directs the expression of said VipR regulator.

The Sigma A-specific −10 box and preferred embodiments of first promoter of SEQ ID No4 are as previously defined.

Preferably the Bacillus strain is chosen among Bacillus thuringiensis, Bacillus cereus, Bacillus weihenstephanensis, Bacillus subtilis, Bacillus megaterium, Bacillus brevis, and more preferably Bacillus thuringiensis.

In a preferred embodiment, the protein of interest may be selected in the group consisting of Vip3A (SEQ ID No10), Cry1I (SEQ ID No11) and Cry2Ab (SEQ ID No12), preferably the protein of interest is selected in the group consisting of Vip3A and Cry1I and more preferably the protein of interest is Vip3A.

The first promoter may be selected in the group consisting of Pvip3 (SEQ ID No15 or SEQ ID No3), Pamidase-1 (SEQ ID No6), Pamidase-2 (SEQ ID No7), Pcry1I (SEQ ID No8 or SEQ ID No76), Pcry2Ab (SEQ ID No9), Pamidase_{pBMB65} (SEQ ID No72 or SEQ ID No73), Pamidase_{pBMB95} (SEQ ID No65 or SEQ ID No74) and Pamidase_{pHT73} (SEQ ID No66 or SEQ ID No75), preferably the promoters are selected in the group consisting of Pvip3 and Pcry1I and more preferably the promoter is Pvip3.

Preferably, the expression system of a protein of interest also comprises:
a stabilizing sequence of the mRNA positioned downstream of the promoter and upstream of the sequence of the gene encoding the protein of interest; preferably, it is STAB-SD of sequence SEQ ID No13; preferably, the STAB-SD sequence is downstream of the transcription +1 and at a position between about 100 and 500 nucleotides upstream of the ribosomal binding site, preferably between 100 and 300 and more preferably between 100 and 150;
a terminator sequence of cry1Ac gene, designated Tcry1Ac, with at least 90% identity with the SEQ ID No14; this sequence is positioned downstream of the sequence of the gene encoding the protein of interest.

However the choice of these sequences should not be considered as limiting because person skilled in the art to can substitute these sequences with sequences with equivalent functions.

In specific embodiments, the expression system of a protein of interest comprises:
(i) at least one polynucleotide sequence encoding a protein of interest; said protein of interest may be selected in the group consisting of Vip3A, Cry1I and Cry2Ab, preferably Vip3A and Cry1I and more preferably Vip3A;
(ii) at least one first promoter comprising a sequence:

(SEQ ID Nº 4)
5′ TTC-N-N-N-N-AT-N-G-N-N-GAA-N-
N-TAT-N-T-N-T-N-CTTT 3′,

wherein N is A or T or C or G; and
wherein said sequence of SEQ ID No4 is positioned upstream between 15 and 21 nucleotides of the Sigma A-specific −10 box preceding the start codon of the gene encoding a protein of interest and more preferably 18 nucleotides;
said first promoter may be selected in the group consisting of Pvip3, Pamidase-1, Pamidase-2, Pcry1I, Pcry2Ab, Pamidase_{pBMB65}, Pamidase_{pBMB95} and Pamidase_{pHT73}, preferably Pvip3 and Pcry1I and more preferably Pvip3;
(iii) the vipR regulator gene;
(iv) the second promoter PvipR;
(v) optionally a stabilizing sequence of the mRNA, preferably STAB-SD, and/or the terminator sequence of the cry1Ac gene, Tcry1Ac.
or
(i) at least one polynucleotide sequence encoding a protein of interest; said protein of interest may be selected in the group consisting of Vip3A, Cry1I and Cry2Ab, preferably Vip3A and Cry1I and more preferably Vip3A;
(ii) at least one first promoter comprising a sequence:

(SEQ ID Nº 4)
5′ TTC-N-N-N-N-AT-N-G-N-N-GAA-N-
N-TAT-N-T-N-T-N-CTTT 3′,

wherein N is A or T or C or G; and
wherein said sequence of SEQ ID No4 is positioned upstream between 15 and 21 nucleotides of the Sigma A-specific −10 box preceding the start codon of the gene encoding a protein of interest and more preferably 18 nucleotides;
said first promoter may be selected in the group consisting Pvip3, Pamidase-1, Pamidase-2, Pcry1I, Pcry2Ab, Pamidase_{pBMB65}, Pamidase_{pBMB95} and Pamidase_{pHT73}, preferably Pvip3 and Pcry1I and more preferably Pvip3;
(iii) the vipR regulator gene;
(iv) the second promoter PvipR;
(v) the STAB-SD sequence.
or
(i) at least one polynucleotide sequence encoding a protein of interest; said protein of interest may be selected in the group consisting of Vip3A, Cry1I and Cry2Ab, preferably Vip3A and Cry1I and more preferably Vip3A;
(ii) at least one first promoter comprising a sequence:

(SEQ ID Nº 4)
5′ TTC-N-N-N-N-AT-N-G-N-N-GAA-N-
N-TAT-N-T-N-T-N-CTTT 3′,

wherein N is A or T or C or G; and
wherein said sequence of SEQ ID No4 is positioned upstream between 15 and 21 nucleotides of the Sigma A-specific −10 box preceding the start codon of the gene encoding a protein of interest and more preferably 18 nucleotides;
said first promoter may be selected in the group consisting Pvip3, Pamidase-1, Pamidase-2, Pcry1I, Pcry2Ab, Pamidase_{pBMB65}, Pamidase_{pBMB95} and Pamidase_{pHT73}, preferably Pvip3 and Pcry1I and more preferably Pvip3;
(iii) the vipR regulator gene;
(iv) the second promoter PvipR;
(v) the terminator sequence of the cry1Ac gene, Tcry1Ac.
or
(i) at least one polynucleotide sequence encoding a protein of interest; said protein of interest may be selected in the group consisting of Vip3A, Cry1I and Cry2Ab, preferably Vip3A and Cry1I and more preferably Vip3A;
(ii) at least one first promoter comprising a sequence:

(SEQ ID Nº 4)
5′ TTC-N-N-N-N-AT-N-G-N-N-GAA-N-
N-TAT-N-T-N-T-N-CTTT 3′,

wherein N is A or T or C or G; and
wherein said sequence of SEQ ID No4 is positioned upstream between 15 and 21 nucleotides of the Sigma A-specific −10 box preceding the start codon of the gene encoding a protein of interest and more preferably 18 nucleotides;
said first promoter may be selected in the group consisting Pvip3, Pamidase-1, Pamidase-2, Pcry1I, Pcry2Ab, Pamidase_{pBMB65}, Pamidase_{pBMB95} and Pamidase_{pHT73}, preferably Pvip3 and Pcry1I and more preferably Pvip3;
(iii) the vipR regulator gene;
(iv) the second promoter PvipR;
(v) the STAB-SD sequence and the terminator sequence of the cry1Ac gene, Tcry1Ac.

The components of the expression system and of the expression system of a protein of interest may be inserted into at least one vector such as a plasmid.

Preferably, the plasmids exhibit a high segregational stability (for example, the vector must stain in the bacterial strain for at least 25 generations) and/or a structural stability (the vector does not recombine with the chromosomal DNA of the bacteria into which it is incorporated); it may be chosen for example among a high copy number vector pHT304, pHT315 and pHT370 or a low copy number vector chosen for example among pHT73 or pBMB299.

The several components of the expression systems do not need to be harbored by the same vector. Accordingly, the VipR regulator and its promoter may be located on a first vector and the protein of interest and the promoter regulated by VipR may be located on a second vector.

In another embodiment, the expression system of a protein of interest may be used to produce at least two proteins of interest. Here again, the components of the expression system can be located on the same vector or on different vectors. Preferably, the expression system of a protein of interest comprises sequences of several genes encoding cytotoxic proteins, and more preferably this expression system comprises the gene encoding Vip3A and the genes encoding at least one Cry toxin.

The vectors according to the invention can be introduced into the host bacterium according to techniques known to those skilled in the art; in particular, the transformation of the host bacterium can be carried out by electroporation (Lereclus et al., 1989) or by heterogramic conjugation (Tieu-Cuot et al., 1987). The expression system can also be introduced on the bacterial chromosome or on a resident plasmid by homologous recombination (Lereclus et al., 1992).

The present invention further relates to a recombinant strain of Bacillus, preferably the strain of Bacillus is non sporulating.

In order to suppress the sporulation activity of a strain of the genus Bacillus, it is possible to inactivate gene essential to sporulation such as the genes involved in the expression of the transcriptional regulator Spo0A responsible for the initiation of sporulation or in expression of the sigma sporulation factors SigE, SigF, SigH and SigK; preferably, the inactivated gene is spo0A, sigE, sigH or sigK. The inactivation of these genes can be obtained by interrupting or modifying the coding sequence, or by deleting all or part of the gene. The deletion is obtained by double crossing-over between the adjacent regions located upstream and downstream of the gene, using plasmids whose replication is heat-sensitive, for example the plasmids pRN5101 (Lereclus et al., 1992) or pMAD (Arnaud et al., 2004), and using the protocols described in these articles. The deletion of the spo0A gene (designated Δspo0A) has a very early effect, as soon as the bacteria enter stationary phase, in particular preventing the bacteria from engaging in the sporulation process (Lereclus et al., 1995). Deletion of the sigE gene (designated ΔsigE) has a later effect, blocking the progression of the sporulation process (Bravo et al., 1996). In both cases, the Bacillus strain can not sporulate, dies and contains almost only the protein of interest.

Preferably, the strain used is Bt kurstaki HD73 Δspo0A or Bt 407 ΔsigE.

The protein of interest produced in the non sporulating Bacillus strain are encapsulated in the dead bacteria, facilitating the recovery of this protein.

According to another embodiment of the invention, the Bacillus strain of the invention does not express amidase 1 or amidase 2 for example by deleting corresponding genes, or expresses inactive Amidase 1 or Amidase 2 proteins.

The present invention also relates to a method for preparing a recombinant strain of Bacillus comprising the steps of:
a—preparing a vector comprising the expression system;
b—transforming said strain with the vector obtained at step (a).

The culture of the bacterial strain is carried out on a culture medium containing at least one source of nitrogen and glucose in suitable concentrations at a temperature preferably between 25 and 35° C., preferably the temperature is about 30° C.; for example, the culture medium is LB medium.

The present invention also relates to a method for producing a protein of interest comprising the steps of:
a—preparation of the bacterial strain according to the invention;
b—culture of said bacterial strain in stationary phase, preferably at a temperature ranging from 25 to 37° C., preferably from 30 to 35° C.; and
c—optionally, purification of said protein of interest.

Purification of the protein of interest can be achieved by centrifugation; in addition, the methods of exclusion chromatography, ion exchange chromatography or even affinity chromatography can be implemented.

According to one embodiment, the protein of interest produced is an insecticidal protein of B. thuringiensis.

It is known that such proteins can be used as a biopesticide in preparations conventionally containing the insecticidal protein in crystal form and bacterial spores, to control crop pests as well as disease vectors such as mosquitoes. In particular, they may be proteins of the Cry family (crystal proteins) or proteins of the Cyt family (cytolytic insecticidal toxins) or Vip3 proteins (toxins active against lepidopteran insects) and more preferentially the proteins of Vip3 proteins or of the Cry family.

The present invention thus relates to the use of a recombinant strain of Bacillus expressing insecticidal proteins according to the invention as a biopesticide.

After their expression, the proteins are protected from degradation by the bacterial membrane which constitutes the envelope of the bacterial sac. In addition, the non-sporulation of the bacterium gives the invention the advantage of not disseminating the spores in the environment.

### FIGURES

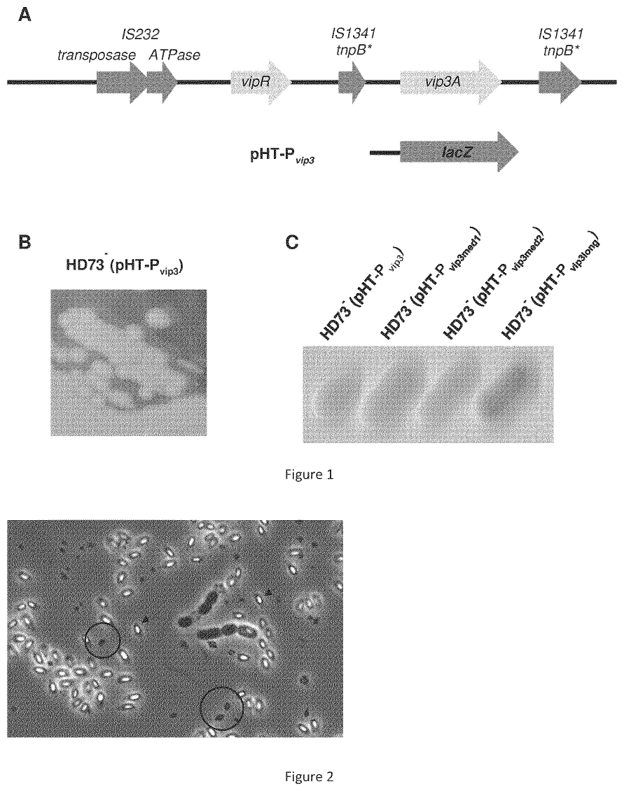

FIG. 1—Vip3A is not expressed in the HD73− strain.
A—Genetic organization of Bt HD1 pBMB299 plasmid region containing the vip3A gene (NZ_CP004876.1). The asterisk indicates truncated genes containing nonsense mutations. Schematics representing the DNA fragment screened for its ability to produce a transcriptional activity.
B—The HD73− (pHT-Pvip3) strain was isolated on LB X-gal (50 μg/mL) plates and grown at 37° C. for 24 h.
C—The HD73− strains carrying the pHT-Pvip3, the pHT-Pvip3 med1, the pHTPvip3med2, or the pHT-Pvip3long were isolated on LB X-gal (50 μg/mL) plates and grown at 37° C. for 24 h.

FIG. 2—Conjugative transfer of the pBMB299 into the HD73− strain allows the cells to produce Cry crystals.
Phase contrast microscope images of Bt HD73⁻ SmR (pBMB299) sporulating cells. Cells were grown on HCT plates for 4 days at 30° C. Spores are the refringent structures indicated with arrows. The crystals are indicated using circles.

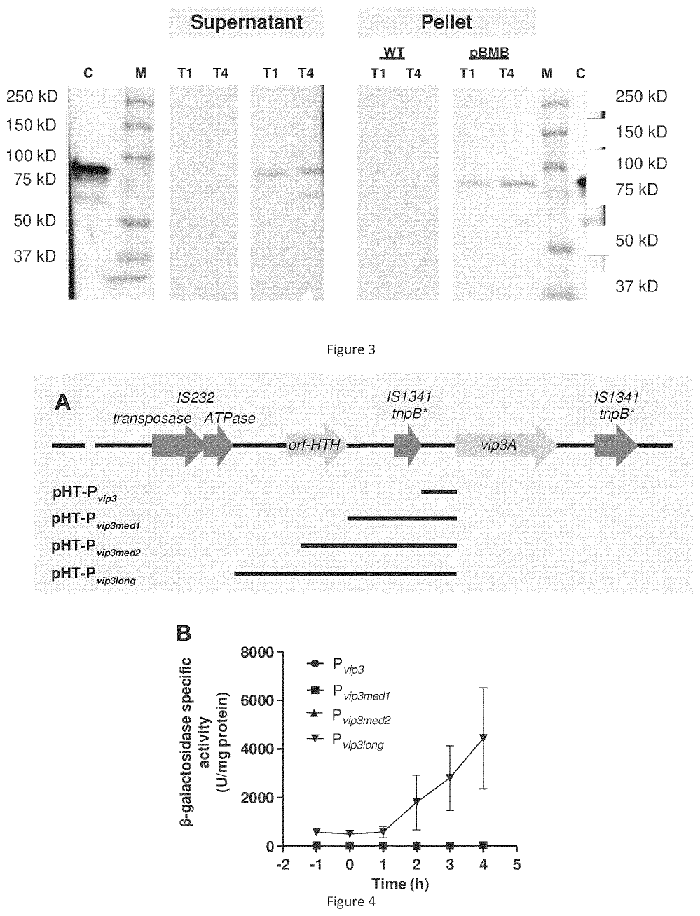

FIG. 3—Expression of vip3A in the B. thuringiensis kurstaki HD73 Cry− strain.
Western blot analysis of Vip3Aa production in the B. thuringiensis HD73− -WT- and HD73− SmR (pBMB299)-pBMB-strains. Strains were cultured in LB medium at 37° C. Samples were collected 1 h (T1) and 4 h (T4) after the entry into stationary phase. The supernatant and cell pellet proteins were prepared as described in M&M. 20 μg of proteins were loaded in each well. 0.1 μg of purified Vip3Aa was used as a control. C—control, M—protein molecular weight marker.

FIG. 4—Characterization of the DNA region required for vip3A gene expression.
A—Schematics representing the genes located upstream and downstream of the vip3A ORF and the DNA fragments screened for their ability to produce a transcriptional activity. The asterisk indicates a gene containing nonsense mutations.
B—The β-galactosidase activity of B. thuringiensis HD73− strains harboring the pHT-Pvip3, pHT-Pvip3med1, pHT-Pvip3med2 or the pHT-Pvip3long plasmid. The activity was assayed when B. thuringiensis HD73⁻ strains were grown in LB at 37° C. Data are the mean±SEM, n=2.

FIG. 5—Characterization of the vip3A promoter elements.
Schematic representation of the vip3A locus (SEQ ID No50). The transcriptional start site is indicated with an arrow at position −403 relative to the vip3A start codon. The asterisk indicates a gene containing nonsense mutations. A focus on the DNA sequence that contains the vip3A promoter elements is given below. The TSS identified using RACE PCR is indicated in bold. The DNA sequence corresponding to the putative −10 box is italicized. The palindromic sequences that are predicted to form a hairpin structure by the mFold software are underlined. The RNA used for the RACE-PCR was prepared from B. thuringiensis HD1 cells grown in LB medium and collected at T2.

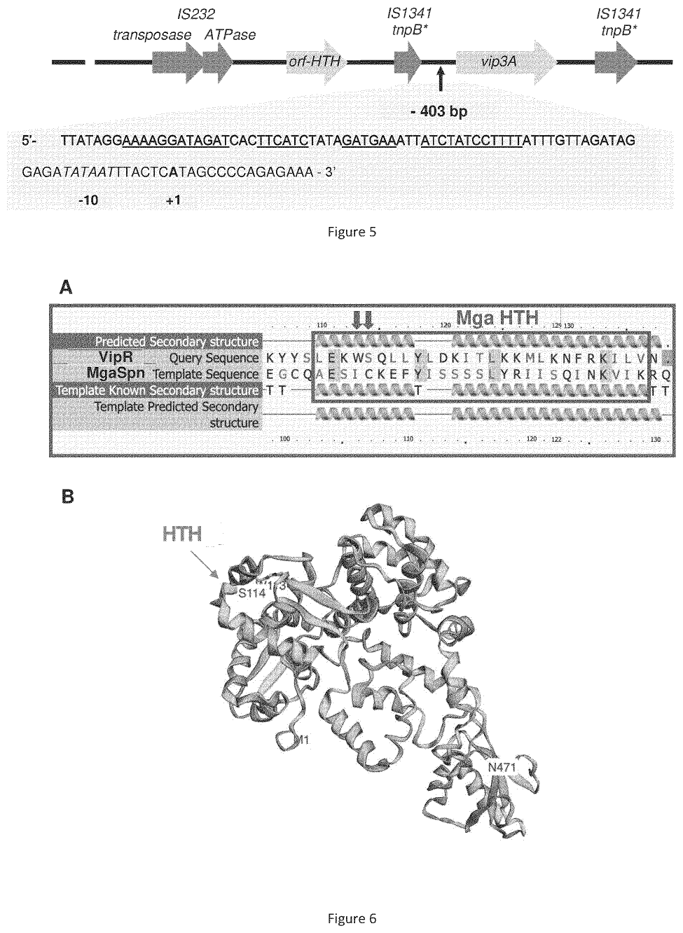

FIG. 6—Mutation of the VipR HTH.
A—Alignment of the VipR (SEQ ID No51) and MgaSpn (SEQ ID No43) protein sequences. The sequence corresponding to the HTH domain of S. pyogenes Mga of is boxed. Arrows point to the 2 amino acids that were selected to be mutated.
B—3D model of the VipR structure modelized by the Phyre2 webserver. The W113 and S114 AA are indicated in pink. The M1 and N471 amino acids at the N- and C-termini are indicated

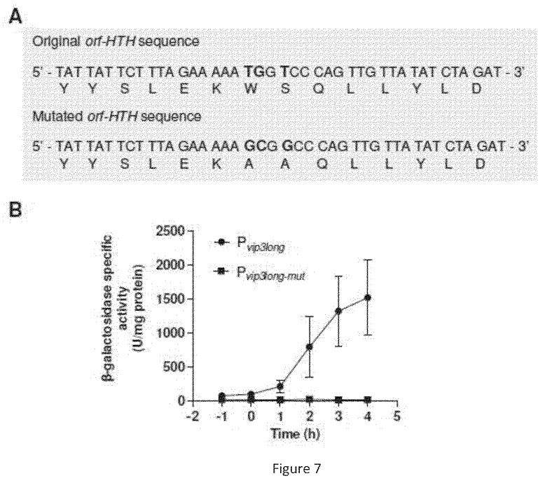

FIG. 7—Mutations in the orf-HTH gene abolish the Pvip3long transcriptional activity.
A—Codons specifying the amino acids W113 and S114 were modified to each code for an alanine. Mutated bases are indicated in bold.
B—β-galactosidase activity of the HD73⁻ strains carrying the pHT-Pvip3long or the pHT-Pvip3long-mut plasmid. Bacteria were grown in LB at 37° C. Time 0 corresponds to the entry of the bacteria into stationary phase. Data are the mean±SEM, n=2

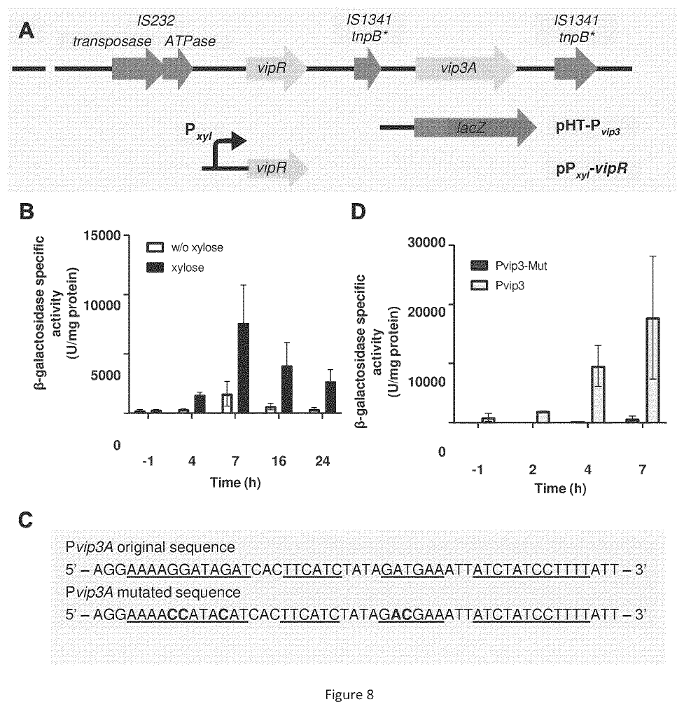

FIG. 8—Activation of the promoter of vip3 by VipR.
A—Schematic representation of the constructs used to study the regulation of the vip3 gene. The asterisk indicates a gene containing nonsense mutations.
B—β-galactosidase activity of the B. thuringiensis HD73⁻ (pHT-Pvip3, pPxyl-vipR) cells grown in the absence or in the presence of xylose (20 mM). Bacteria were cultured in LB at 37° C.
C—DNA sequence of the vip3A promoter (SEQ ID No52). Bases forming the palindrome are underlined. Mutated bases are indicated in bold (SEQ ID No48).
D—β-galactosidase activity of the B. thuringiensis HD73⁻ (pHT-Pvip3, pPxyl-vipR) and HD73⁻ (pHT-Pvip3-mut, pPxyl-vipR) cells grown in the presence of xylose (20 mM). Bacteria were cultured in LB at 30° C. Time 0 corresponds to the entry of the bacteria into the stationary phase. Xylose was added at T-1. Data are the mean±SEM, n=2.

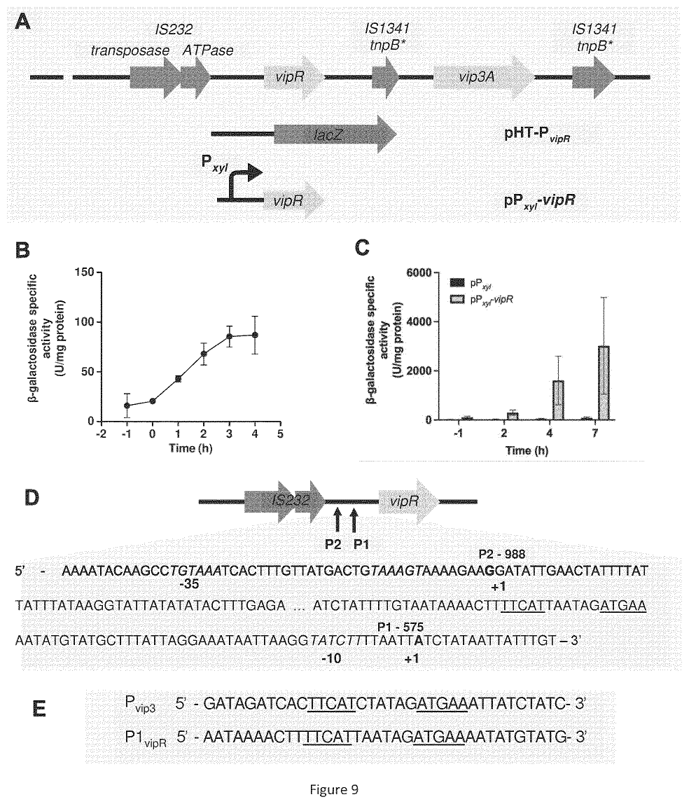

FIG. 9—VipR is an autoregulated transcriptional activator.
A—Schematic representation of the constructs used to study the regulation of the vipR gene.
B—β-galactosidase activity of the B. thuringiensis HD73− (pHT-PvipR) cells grown in LB at 37° C.
C—β-galactosidase activity of the HD73− (pHT-PvipR, pPxyl-vipR) and HD73− (pHT-PvipR, pPxyl) cells grown in the presence of xylose (20 mM) at 30° C. Time 0 corresponds to the entry of the bacteria into the stationary phase. Xylose was added at T-1. Data are the mean±SEM SEM, n=2.
D—Schematic representation of the vipR genetic organization (SEQ ID No53). The transcriptional start sites are indicated with an arrow. P1 and P2 are located at position −575 and −988 to the vip3A start codon, respectively. A focus on the DNA sequence that contains the vipR promoter elements is given. The TSS identified using RACE-PCR are indicated in bold. The DNA sequence corresponding to the putative −10 box is italicised. The palindromic sequence in P1 is underlined. The RNA used for the RACE-PCR was prepared from HD73⁻ pHT-Pvip3long cells grown in LB medium and collected at T2.
E—Alignment of the DNA sequences of the vipR (SEQ ID No55) and vip3A (SEQ ID No54) promoters highlighting the conserved palindromic sequences.

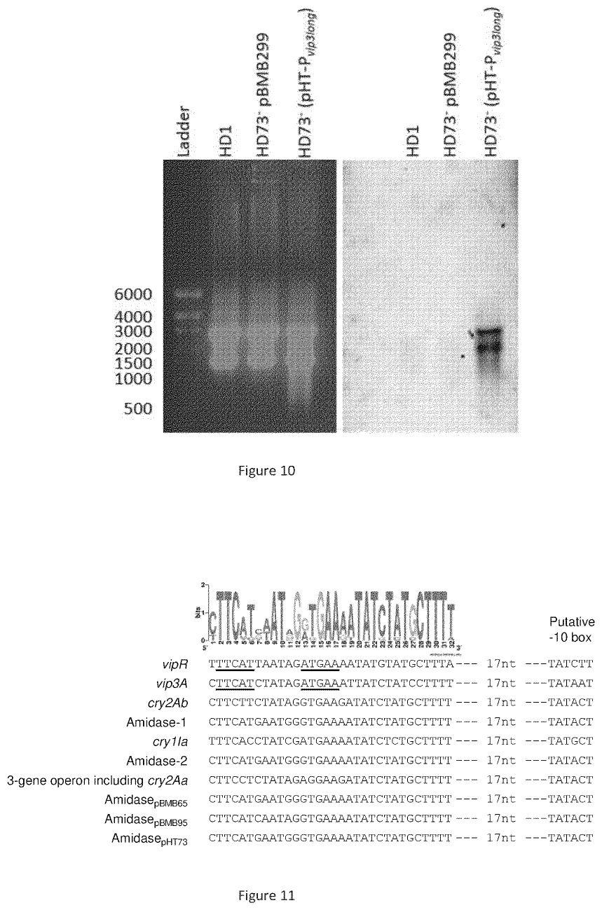

FIG. 10—Northern Blot analysis of vipR transcription indicates the presence of two transcripts in the HD73⁻ (pHT-Pvip3long) strain.
10 μg RNAs were separated on a 1% agarose gel and transferred overnight by capillarity on a nylon membrane (GE Healthcare, #RPN303B) in SSC 10× Buffer. RNAs were then UV-crosslinked to the membrane using a Stratalinker apparatus. Generation and incubation of the membrane with DNA dig-labeled probes was performed using the Dig-High Prime DNA Labeling and Detection Starter Kit II (Roche, #11585614910), following manufacturer's instructions. Primers used to generate the DNA probes are NB-vipR-fw (GCATAAGTTCAATTATATGCGAATTG, SEQ ID No41) and NB-vipR-fw (TTTCAGAAGATATTGTTTGGAATAAATGT, SEQ ID No42). Membranes were washed twice in 2×SSC 0.1% SDS and once in 1×SSC 0.1% SDS, for 10 min at 65° C. Revelation was done according to the kit instructions using a Chemidoc System (Bio-Rad). Numbers indicated at the left indicate the size (in bases) of the single-stranded RNA transcripts used in the RiboRuler High Range RNA Ladder (Thermo Scientific)

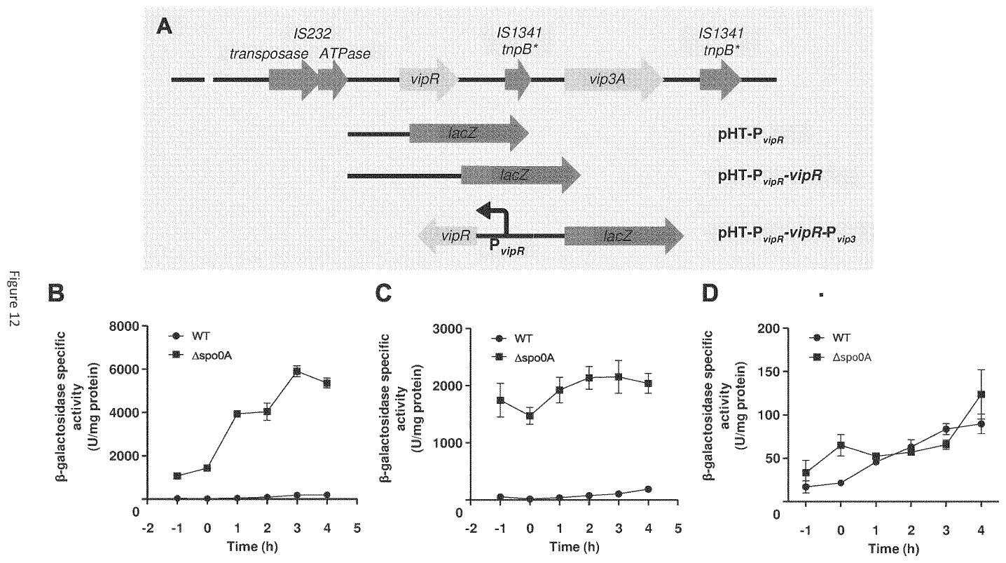

FIG. 11—Alignment of the conserved sequences found in the pBM299 plasmid.
The name of the gene putatively controlled by VipR (SEQ ID No49, 56-64) is indicated on the left. Distance between the putative −10-box and the last nucleotide of the conserved motif is indicated. A consensus is shown on top as a sequence logo in which the height of the letters in bits is proportional to their frequency.

FIG. 12—Expression of vip3A is increased in the Bt HD73 Cry⁻ Spo0A⁻.
A—Schematic representation of the constructs used to study the regulation of the vip3 and vipR genes in the sporulation mutant strain.
B—β-galactosidase activity of the B. thuringiensis HD73− (pHT-PvipR-vipR) and HD73− Spo0A−(pHT-PvipR-vipR) cells.
C—β-galactosidase activity of the HD73− (pHT-PvipR-vipR-Pvip3) cells and HD73− Spo0A−(pHT-PvipR-vipR-Pvip3).
D—β-galactosidase activity of the HD73− (pHT-PvipR) and HD73-Spo0A−(pHT-PvipR) cells. Strains were grown in LB at 37° C. Time 0 corresponds to the entry of the bacteria into the stationary phase. Data are the mean±SEM, n=at least 2.

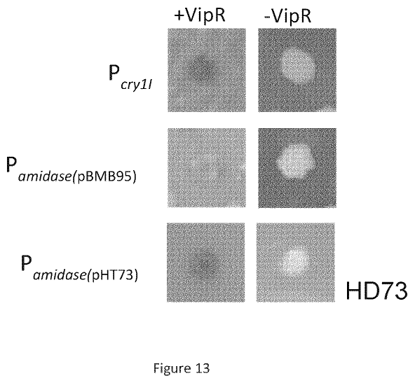

FIG. 13—Expression of lacZ gene fused to three different promoters in a presence or not of vipR gene.
Colonies of the B. thuringiensis kurstaki HD73 strain harboring a transcriptional fusion between the promoter of the cry1I or amidase genes and the lacZ gene, in the presence (left) or in the absence (right) of the vipR gene. Bacteria were grown on LB medium (top and middle panels) or HCT-glucose-Xylose medium containing X-gal (bottom panel).

### EXAMPLE

### Materials and Methods

### Strains and Plasmids Construction.

The acrystalliferous strain B. thuringiensis HD73 Cry⁻ belonging to serotype 3 (22) was used as a heterologous host throughout this study and was designated as HD73⁻. Escherichia coli strain DH5α was used as the host strain for plasmid construction. E. coli strain BL21 λDE3 (Invitrogen) was used to produce the Vip3Aa protein. E. coli strain ET12567 (51) was used to prepare demethylated DNA prior to electroporation in B. thuringiensis (52, 53). The plasmids and bacterial strains used in the study are listed in Table 1 and 2, respectively. Bacteria were routinely grown in LB medium at 37° C., and B. thuringiensis cells were cultured at 30° C. when indicated. Time 0 was defined as the beginning of the transition phase between the exponential and stationary phases of B. thuringiensis growth. The following concentrations of antibiotics were used for B. thuringiensis selection: erythromycin 10 μg/mL, streptomycin 200 μg/mL and kanamycin 200 μg/mL. For E. coli selection, kanamycin 20 μg/mL and ampicillin 100 μg/mL were used. When needed, 20 mM of xylose was added to the culture.

TABLE 1
Plasmids used

| Short name | Plasmid name | Descriptions |
|---|---|---|
| pHT-Pᵥᵢₚ₃ | pHT304.18Z- | The promoter of the vip3Aa gene was PCR amplified using |
| | Pᵥᵢₚ₃ | genomic DNA of Bt HD1 and primers Pvip3-fw- |
| | | HindIII/Pvip3-rev-BamHI. The 709 bp DNA fragment was |
| | | ligated between the BamHI-HindIII sites of the |
| | | pHT304.18Z (24). |
| pHT1618K | pHT16.18K | E. coli/B. thuringiensis high-copy number shuttle vector |
| | | conferring resistance to the kanamycin (46) |
| pBMB299 | pBMB299 | The native insecticidal plasmid of the Bt HD1 strain. It |
| | | encodes notably the vip3Aa gene and 4 cry genes, cry2Ab, |
| | | cry2Aa cry1Aa and cry1Ia. |
| pHT-P_{vip3med1} | pHT304.18Z- | A 2454 bp fragment encompassing the promoter of the |
| | P_{vip3med1} | vip3Aa gene, the transposase gene and the intergenic |
| | | regions between vipR and the transposase gene was PCR |
| | | amplified using genomic DNA of Bt HD1 and primers pair |
| | | Pvip3med1-fw-HindIII/Pvip3-rev-BamHI. The DNA |
| | | fragment was ligated between the BamHI-HindIII sites of |
| | | pHT304.18Z (24). |
| pHT-P_{vip3med2} | pHT304.18Z- | A 3701 bp fragment containing the promoter of the |
| | P_{vip3med2} | vip3Aa gene, the transposase gene, the intergenic regions |
| | | between vipR and the transposase gene, and 355 bp of |
| | | vipR was PCR amplified using genomic DNA of Bt HD1 and |
| | | the primers Pvip3med2-fw-HindIII/Pvip3-rev-BamHI. The |
| | | DNA fragment was ligated in the BamHI-HindIII sites of |
| | | pHT304-18Z (24). |
| pHT-P_{vip3long} | pHT304.18Z- | A 5096 bp fragment containing the promoter of vip3Aa |
| | P_{vip3long} | gene, the transposase gene, the intergenic region |
| | | between vipR and transposase gene, vipR and the |
| | | intergenic region upstream the vipR orf was PCR amplified |
| | | using genomic DNA of Bt HD1 and the primers Pvip3long- |
| | | fw-HindIII/Pvip3-rev-BamHI. This DNA fragment was |
| | | ligated in the BamHI-HindIII sites of pHT304-18Z (24). |
| pHT-P_{vip3long-mut} | pHT304.18Z- | Point mutations were introduced into the vipR coding |
| | P_{vip3long-mut} | sequence to replace the W113 and S114 residues by |
| | | alanine by overlapping PCR amplification using the primer |
| | | pair vipR-mut-fw/vipR-mut-rev and the pHT304.18Z- |
| | | Pvip3long plasmid as template. |
| pP_{xyl} | pHT16.18K-P_{xylA} | The pHT16.18K plasmid that contains the xylR repressor |
| | | gene and the promoter of the xylA gene to allow gene |
| | | expression upon xylose induction (46). |
| pP_{xyl}-vipR | pHT16.18K-P_{xyl}- | A 1496 bp fragment containing the vipR coding sequence |
| | vipR | was PCR amplified using genomic DNA of Bt HD1 as |
| | | template and the primer pair vipR-fw/PvipR-Rev- |
| | | Smal_BamHI. The DNA fragment was ligated in the BamHI |
| | | site of pHT16.18K generating a transcriptional fusion with |
| | | the xylA promoter. |
| pHT-P_{vip3-Mut} | pHT304.18Z- | The Pᵥᵢₚ₃ DNA sequence with 5 mutations designed to |
| | P_{vip3-Mut} | disrupt the palindromic sequence has been synthesized. |
| | | The 709 bp DNA fragment was ligated between the |
| | | BamHI-HindIII sites of the pHT304.18Z (24). |
| pHT-P_{vipR} | pHT304.18Z- | A 1581 bp BamHI-HindIII fragment containing the |
| | P_{vipR} | promoter of vipR and 355 bp of vipR was PCR amplified |
| | | using genomic DNA of Bt HD1 as template and the primer |
| | | pair Pvip3long-fw-HindIII/vipRmed-rev-BamHI. The DNA |
| | | fragment was ligated between the BamHI-HindIII sites of |
| | | pHT304-18Z (24). |
| pHT-P_{vipR}-vipR | pHT304.18Z- | A 1581 bp DNA fragment containing the promoter of vipR |
| | P_{vipR}-VipR Fw | and the vipR coding sequence was PCR amplified using |
| | | genomic DNA of Bt HD1 as template and the primer pair |
| | | Pvip3long-fw-HindIII/vipR-rev-BamHI. This DNA fragment |
| | | was ligated in the BamHI-HindIII sites of pHT304-18Z (24). |
| pHT-P_{vipR}-VipR- | pHT304.18Z- | A 2642 bp DNA fragment containing the promoter of vipR |
| REV | P_{vipR}-VipR REV | and full length of vipR was PCR amplified using genomic |
| | | DNA of Bt HD1 and primer pairs Pvip3long-fw-Pstl/vipR- |
| | | rev-HindIII. This DNA fragment was ligated in the Pstl- |
| | | HindIII sites of pHT304.18Z (24) in the reverse compared |
| | | to lacZ. |
| pHT-P_{vipR}-VipR- | pHT304.18Z- | A 709 bp Pstl-HindIII fragment containing the promoter of |
| Pᵥᵢₚ₃ | P_{vipR}-VipR-Pᵥᵢₚ₃ | vip3A was PCR amplified using genomic DNA of Bt HD1 |
| | | and primer pairs Pvip3-fw-Pstl/Pvip3-rev-BamHI. This |
| | | DNA fragment was ligated in the Pstl-HindIII sites of the |
| | | pHT-P_{vipR}-vipR REV in the same direction as lacZ. |
| pET28aΩvip3A | pET28aΩvip3A | A 2370 bp BamHI-Ndel fragment containing the vip3A |
| | | gene was PCR amplified using genomic DNA of Bt HD1 and |
| | | primer pairs vip3-fw-Ndel/vip3-rev-BamHI. This DNA |
| | | fragment was ligated in the Ndel-BamHI sites of pET28a. |
| | | The protein is produced with a 5′ His-tag. |
| pHT-P_{cry1/} | pHT304.18Z- | The promoter of the cry1la gene was PCR amplified using |
| | P_{cry1/} | genomic DNA of Bt HD1 and primers Pcry1la-F/Pcry1la-R. |
| | | The 381 bp DNA fragment was ligated between the |
| | | BamHI-HindIII sites of the pHT304.18Z (24). |
| pHT- | pHT304.18Z- | The promoter of the amidase gene was PCR amplified |
| P_{amidase(pBMB95)} | P_{amidase(pBMB95)} | using genomic DNA of Bt HD1 and primers Pamidase- |
| | | F/Pamidase-R. The 199 bp DNA fragment was ligated |
| | | between the BamHI-HindIII sites of the pHT304.18Z (24). |
| pHT-P_{amidase(pHT73)} | pHT304.18Z- | The promoter of the amidase gene was PCR amplified |
| | P_{amidase(pHT73)} | using genomic DNA of Bt HD73 and primers Pamidase2-F- |
| | | HdIII/Pamidase-R. The 343 bp DNA fragment was ligated |
| | | between the BamHI-HindIII sites of the pHT304.18Z (24). |

TABLE 2
Strains used

| | | Antibiotic |
|---|---|---|
| Strain | Description | resistance |
| HD73⁻ | B. thuringiensis kurstaki HD73 cured of the plasmid pHT73 | — |
| | carrying the cry1Ac gene. This Cry strain does not produce | |
| | any crystal protein. | |
| HD1 | B. thuringiensis kurstaki HD1 strain. The strain harbors the | — |
| | insecticidal plasmid pBMB299. | |
| HD73⁻ Sm^{R} | Strain HD73⁻ which is resistant to the streptomycin. This strain | Strep |
| | was used as the recipient strain for the pBMB299 and | |
| | pHT16.18K plasmids in the conjugation experiment. Lab | |
| | stock. | |
| HD1 (pBMB299, | Strain HD1 transformed with the pHT16.18K plasmid. This | Kan |
| pHT16.18K) | strain was used as the donor strain in the conjugation | |
| | experiment. | |
| HD73⁻ Sm^{R} | Strain HD73⁻ Sm^{R} ex-conjugating. This strain received the | Strep, Kan |
| (pBMB299, | pBMB299 and pHT16.18K plasmids γ conjugation from the | |
| pHT16.18K) | HD1 strain. | |
| HD73⁻ Sm^{R} | Strain HD73⁻ Sm^{R} harboring the pBMB299. The strain was | Strep |
| (pBMB299) | cured from the pHT16.18K plasmid. | |
| HD73⁻ (pHT-Pᵥᵢₚ₃) | Strain HD73⁻ harboring the pHT-Pᵥᵢₚ₃ plasmid to measure the | Ery |
| | activity of the vip3A promoter. | |
| HD73⁻ (pHT-P_{vip3med1}) | Strain HD73⁻ harboring the pHT-P_{vip3med1} plasmid to measure | Ery |
| | the transcriptional activity of the P_{vip3med1} DNA fragment. | |
| HD73⁻ (pHT-P_{vip3med2}) | Strain HD73⁻ harboring the pHT-P_{vip3med2} plasmid to measure | Ery |
| | the transcriptional activity of the P_{vip3med2} DNA fragment. | |
| HD73⁻ (pHT-P_{vip3long}) | Strain HD73⁻ harboring the pHT-P_{vip3long} plasmid to measure | Ery |
| | the transcriptional activity of the P_{vip3long} DNA fragment. | |
| HD73⁻ (pHT-P_{vip3long-} | Strain HD73⁻ harboring the pHT-P_{vip3long-mut} plasmid to measure | Ery |
| ₘᵤₜ) | the transcriptional activity of the mutated P_{vipslong-mut} DNA | |
| | fragment. | |
| HD73⁻ | Strain HD73⁻ expressing vipR under the control of the xylose- | Kan |
| (pP_{xyl}-vipR) | inducible promoter of xylA. | |
| HD73⁻ | Strain HD73⁻ expressing vipR under the control of the xylose- | Kan, Ery |
| (pP_{xyl}-vipR, pHT- | inducible promoter of xylA and used to measure the | |
| Pᵥᵢₚ₃/pHT-P_{vipR/}pHT- | transcriptional activity of the vip3A, vip3A-Mut and vipR | |
| P_{vip3-Mut}) | promoter using the lacZ reporter gene in the presence of | |
| | VipR. | |
| HD73⁻ (pHT-P_{vip3-Mut}) | Strain HD73⁻ harboring the pHT-P_{vip3-Mut} plasmid to measure | Ery |
| | the transcriptional activity of the vip3A promoter when the | |
| | palindromic sequence is disrupted. | |
| HD73⁻ (pHT-P_{vipR}) | Strain HD73⁻ harboring the pHT-P_{vipR} plasmid to measure the | Ery |
| | transcriptional activity of the vipR promoter. | |
| HD73⁻ Δspo0A (pHT- | Strain HD73⁻ Δspo0A harboring the pHT-P_{vipR} plasmid to | Ery |
| P_{vipR}) | measure the transcriptional activity of the vipR promoter. | |
| HD73⁻ (pHT-P_{vipR}- | Strain HD73⁻ harboring the pHT-P_{vipR}-vipR plasmid to measure | Ery |
| vipR) | the transcriptional activity of the P_{vipR} DNA fragment in the | |
| | presence of VipR. | |
| HD73⁻ Δspo0A (pHT- | Strain HD73⁻ Δspo0A harboring the pHT-P_{vipR}-vipR plasmid to | Ery |
| P_{vipR}-VipR) | measure the transcriptional activity of the P_{vipR} DNA fragment | |
| | in the presence of VipR. | |
| HD73⁻ (pHT-P_{vipR}- | Strain HD73 Cry⁻ harboring the pHT-P_{vipR}-vipR-Pᵥᵢₚ₃ plasmid to | Ery |
| vipR-Pᵥᵢₚ₃) | measure the transcriptional activity of the vip3A promoter in | |
| | the presence of a vipR expression cassette. | |
| HD73⁻ Δspo0A (pHT- | Strain HD73⁻ Δspo0A harboring the pHT-P_{vipR}-vipR-Pᵥᵢₚ₃ | Ery |
| P_{vipR}-VipR-Pᵥᵢₚ₃) | plasmid to measure the transcriptional activity of the vip3A | |
| | promoter in the presence of a vipR expression cassette. | |
| BL21 (vip3) | E. coli BL21(DE3) strain harboring the pET28aΩvip3A plasmid, | Kan |
| | used to produce de Vip3Aa protein. | |
| HD73− (pPxyl-vipR, | Strain HD73− expressing vipR under the control of the xylose- | Kan, Ery |
| pHT- Pcry1I/pHT- | inducible promoter of xylA and used to measure the | |
| Pamidase(pBMB95)/ | transcriptional activity of the cry1I, amidase(pBMB95) and | |
| pHT- | amidase(pHT73) promoter using the lacZ reporter gene in the | |
| Pamidase(pHT73) | presence of VipR. | |
| HD73− (pPxyl, pHT- | Strain HD73− expressing vipR under the control of the xylose- | Kan, Ery |
| Pcry1I/pHT- | inducible promoter of xylA and used to measure the | |
| Pamidase(pBMB95)/ | transcriptional activity of the cry1I, amidase(pBMB95) and | |
| pHT- | amidase(pHT73) promoter using the lacZ reporter gene in the | |
| Pamidase(pHT73) | absence of VipR. | |

### DNA Manipulation.

Plasmids were extracted from E. coli cells by the alkaline lysis method using the Promega DNA Extraction Kit. DNA fragments were purified using Promega Gel and PCR Clean-Up System. Chromosomal DNA was extracted from exponentially growing B. thuringiensis HD1 cells using the Qiagen Puregene Yeast/Bacteria kit. Restriction enzymes and T4 DNA ligase were purchased from New England Biolabs and used following the manufacturer's protocol. The primers used in the study (Table 3) were synthesized by Eurofins Genomics. PCR was performed with a 2720 Thermal cycler (Applied Biosystems) or a Master cycler Nexus X2 (Eppendorf). All the constructs were verified by PCR and sequencing. Sequencing was performed by Eurofins genomics.

TABLE 3
Primers used

| | | Restriction |
|---|---|---|
| Name | Sequence (5′-3′) | sites |
| Pvip3-fw-HindIII (SEQ IQ Nº 16) | CCCAAGCTTGACTGTCCTTCTTATCTTACACG | HindIII |
| Pvip3-rev-BamHI (SEQ IQ Nº 17) | CGGGATCCTTTTCAGCTATTTTTTGTAACAC | BamHI |
| vip3-fw (SEQ IQ Nº 18) | ACATCCTCCCTACACTTTCTAATAC | |
| vip3-rev (SEQ IQ Nº 19) | TCTTCTATGGACCCGTTCTCTAC | |
| GSP1 (SEQ IQ Nº 20) | CAGTGGCAAATCCATA | |
| GSP2 (SEQ IQ Nº 21) | CTTGGTAAGGCTCTTGTGCTT | |
| GSP3 (SEQ IQ N°22) | GTTCATGTTCATCTTCCTTTTCAGCTAT | |
| vipR-GSP1 (SEQ IQ Nº 23) | TGCTATCAATCAAGGTT | |
| vipR-GSP2 (SEQ IQ Nº 24) | GTTGTCGTACTATTGATTTATCTTG | |
| vipR-GSP3 (SEQ IQ Nº 25) | CCATTACATTTCTCCTCCCT | |
| midvipR-GSP3 (SEQ IQ Nº 26) | CGAATCCAAAGAAACTACCATCT | |
| Pvip3long-fw-HindIII | CCCAAGCTTTTTTGTACATGCTTAAACA | HindIII |
| (SEQ IQ Nº 27) | AGC | |
| Pvip3med1-fw-HindIII | CCCAAGCTTACTTAAGGTTTTAGTTC | HindIII |
| (SEQ IQ Nº 28) | | |
| Pvip3med2-fw-HindIII | CCCAAGCTTCGCTTCCCGAAAATTGGG | |
| (SEQ IQ Nº 29) | | |
| vipR-mut-fw (SEQ IQ Nº 30) | TATTATTCTTTAGAAAAAGCGGCCCAGT | |
| | TGTTATATCTAGAT | |
| vipR-mut-rev (SEQ IQ Nº 31) | ATCTAGATATAACAACTGGGCCGCTTTT | |
| | TCTAAAGAATAATA | |
| vipR-Fw (SEQ IQ Nº 32) | GGGGATCCGTTTTGTAGTTAAATGTTACC | BamHI |
| PvipR-Rev-Smal_BamHI | CGGGATCCCGGGTTAGTTAAAAGGGGATA | SmaI, |
| (SEQ IQ Nº 33) | AAACTT | BamHI |
| vipRmed-rev-BamHI | CGGGATCCGATATAACAACTGGGACC | BamHI |
| (SEQ IQ Nº 34) | | |
| vipR-rev-BamHI (SEQ IQ Nº 35) | CGGGATCCTTAGTTAAAAGGGGATAAAACT | BamHI |
| Pvip3long-fw-PstI (SEQ IQ N° 36) | AAAACTGCAGTTTTGTACATGCTTAAACAA | Pstl |
| | GC | |
| vipR-rev-HindIII (SEQ IQ Nº 37) | CCCAAGCTTTTAGTTAAAAGGGGATAAAACT | HindIII |
| Pvip3-fw-PstI (SEQ IQ Nº 38) | AAAACTGCAGGACTGTCCTTCTTATCTTACA | Pstl |
| | CG | |
| Vip3-fw-NdeI (SEQ IQ Nº 39) | GGAATTCCATATGAACAAGAATAATACTAAA | Ndel |
| | TTAAGC | |
| Vip3-rev-BamHI (SEQ IQ Nº 40) | CGGGATCCCGTTACTTAATAGAGACATCGTAAA | BamHI |
| | AATG | |
| Pcry1la-F (SEQ IQ Nº 67) | CCCAAGCTTGTGATGTCCGTTTTACTATGTTAT | HindIII |
| | TTTCTAG | |
| Pcry1la-R (SEQ IQ N° 68) | CGGGATCCACTTATACTATTATTTAATTTTCAG | BamHI |
| | ATATAAT | |
| Pamidase-F (SEQ IQ N° 69) | CCCAAGCTTATCACGAAAAGAAGTTTTTAAGAA | HindIII |
| | CCATTCC | |
| Pamidase-R (SEQ IQ N° 70) | CGGGATCCTATCAGATTATTGGATATGATTTCA | BamHI |
| | ATCTTTC | |
| Pamidase2-F-HdIII | CCCAAGCTTTGTAGAAACTTTGTAATTTGTATG | HindIII |
| (SEQ IQ Nº 71) | AATTGAG | |

### Bioinformatic Analyses.

The DNA sequences of the vip3A plasmid of nine B. thuringiensis strains were compared using blast. 17 genes, among them the vip3A and 3 cry genes, all encoded in the same direction, were defined as an island of insecticidal toxins on the B. thuringiensis HD1 pBMB299 plasmid (NZ_CP004876.1) used as the reference. Each gene of the insecticidal toxins island of the pBMB299 was compared using BLAST (https://blast.ncbi.nlm.nih.gov/Blast.cgi) with the corresponding gene on the vip3A plasmid of the following strains: B. thuringiensis BGSC4C1 (CP015177), CT-43 (CP001910), HD12 (CP014853), HD29 (CP010091), IS5056 (CP004136), L7601 (CP020005), YBT-1520 (CP004861) and YC-10 (CP011350). The sequence of the orf-HTH-encoded protein (WP_000357137.1) was subjected to structural prediction using the Phyre2 software (27) available online and the HHpred server (54) that detects structural homologues. The intergenic region upstream from the vip3A coding sequence was analyzed for the presence of secondary structures in nucleic acid sequences using the Mfold web server (28). The same sequence was also analyzed for the presence of a potential Rho-independent transcription terminator using the ARNold web server (http://rssf.i2bc.paris-saclay.fr/toolbox/arnold/).

### Vip3A Protein Production and Purification.

The BL21 (vip3) strain was grown in LB medium supplemented with 20 μg/ml of kanamycin at 37° C. to an OD600 nm of 0.6. The expression of vip3A was induced by adding 1 mM IPTG and growth was continued for 4 h at 37° C. Bacterial cells were collected by centrifugation and resuspended in 5% of the initial culture volume in the lysis buffer (50 mM Tris, 300 mM NaCl, 7.5% glycerol, pH8.0). The bacteria were treated with 1 mg/ml of lysozyme on ice for 60 min and then lysed by sonication. The suspension was centrifuged at 5095×g for 10 min to remove bacterial debris. The supernatant that contains the Vip3A protein was loaded onto 1 ml of Ni-NTA agarose resin previously equilibrated with the lysis buffer. The resin with bound Vip3A proteins was successively washed with 4 mL of lysis buffer containing 25- and 50-mM imidazole. The Vip3A protein was eluted with 1.5 mL of lysis buffer containing 250- and 500-mM imidazole. To remove the imidazole, the buffer of the protein was exchanged against PBS using a PD-10 column (Sigma). The purified protein was stored at −80° C. before use.

Conjugative Transfer of the vip3A-Encoding Plasmid pBMB299.

The B. thuringiensis HD1 (pHT1618K) strain was used as a pBMB299 plasmid donor strain, and the streptomycin resistant HD73− SmR was used as the recipient strain. The donor and recipient strains were grown in LB at 37° C. until OD600 nm 0.7. Then 5×10⁶ cells of the donor and recipient strain were mixed in 2 ml BHI broth. The mixed bacteria were transferred on a 0.45 am membrane by passing the bacterial suspension through the Swinnex© filter holder. The membrane was then put onto a BHI plate and incubated at 37° C. overnight. The bacteria were collected by scrapping and resuspended in physiological water. The suspension was then diluted and plated on LB plates containing 200 μg/mL kanamycin and streptomycin 200 μg/mL to select the exconjugant bacteria. The presence of the pBMB299 plasmid in colonies that have received the pHT1618K was confirmed by PCR using the primer pair vip3-fw/vip3-rev targeting the vip3A gene. The strain HD73− SmR (pHT1618K, pBMB299) was finally cured of the pHT1618K as described (25). Briefly, the bacterial strain was grown on HCT medium for 3 days at 30° C. until sporulation and spores were plated on LB medium containing 200 μg/mL of streptomycin. Isolated colonies were screened for the loss of kanamycin resistance on plates. The presence of the pBMB299 in HD73-SmR KanS clones was confirmed by PCR using the primer pair vip3-fw/vip3-rev.

### Western Blot Assays.

For western blot, B. thuringiensis HD1 and HD73− SmR (pBMB299) strains were grown in LB medium at 37° C. in agitated cultures. For each time point, a 50 mL volume of culture was collected. Bacteria were separated from the growth medium by centrifugation at 5095×g for 10 min. The bacterial cell pellet was resuspended in the lysis buffer (50 mM Tris, 300 mM NaCl, 7.5% glycerol, pH 8.0). The suspension was then treated with 1 mg/mL of lysozyme on ice for 1 h, and sonicated. The proteins of the culture medium were precipitated according to the following steps: 100 mM of dithiothreitol was added to the suspension to prevent protein oxidation, then 19.62 g of (NH4)₂SO₄ was slowly added to 45 ml of sample to reach 70% saturation (0° C.) and the suspension was incubated overnight with slow agitation at 4° C. The precipitated proteins were collected by centrifugation at 5095×g for 10 min, and resuspended in 200 μL of lysis buffer. The protein concentration of the pellets and of the precipitated supernatant samples was determined using the Bradford method (55). 20 μg of proteins of each sample and 0.05 μg of purified Vip3A were separated using SDS-PAGE on a 7.5% polyacrylamide gel. Gels were either stained with Coomassie brilliant blue or subjected to Western blot assays. For Western blot assays, the proteins were electro-transferred to a PVDF membrane (Immun-Blot© PVDF Membrane, Bio-Rad). The membrane was blocked for 1 h in 5% skimmed milk dissolved in TBS-T buffer (10 mM Tris-HCl, 150 mM NaCl, 0.05% Tween-20, pH 8.0), then treated with anti-Vip3Aa11 polyclonal antibodies (56) diluted 1:100 000 in 5% skimmed milk TBS-T buffer for 1 h at room temperature. The membrane was washed three times with 15 ml of TBS-T buffer, and then treated with 1:20 000 diluted Goat anti-rabbit antibodies (Invitrogen G21234) for 1 h. Membranes were washed three times with TBS-T buffer before being revealed using the SuperSignal™ West PicoChemiluminescent substrate (ThermoScientific) according to the manufacturer's instructions, and imaged using the Chemidoc system (Bio-Rad).

β-galactosidase assay.

The β-galactosidase activity was monitored using a qualitative and quantitative method. For the qualitative assay, cells were streaked on LB plates containing X-gal at a concentration of 50 μg/mL and incubated at 37° C. for the indicated periods of time. The blue coloration reflects the activity of the β-galactosidase. For the quantitative method, strains were precultured in 10 ml of LB medium until OD600 nm 0.6-1.0 from freshly isolated strains on plates. Then, the preculture was used to inoculate 50 ml of LB medium at an OD600 nm=0.005 and bacteria were allowed to grow until being harvested by centrifugation at the indicated time points. The activity was measured as previously described and expressed as units per milligram of protein (57).

mRNA Extraction and Determination of the vip3a and vipR Transcriptional Start Sites.

RNA was extracted from the B. thuringiensis HD1 strain grown at 37° C. in LB medium and harvested at T2. RNA samples were prepared as described (7) and stored at −80° C. before use. RACE-PCR was performed using the 5′ RACE System (Invitrogen, cat: 18374058) according to the instruction manual. The cDNA was generated using the specific primer GSP1. The subsequent PCR steps were realized with the nested gene-specific GSP2 and GSP3 primers. The transcription start site of vip3A was determined by sequencing the PCR product using GSP3. For vipR transcription start sites determination, RNA was extracted from the B. thuringiensis HD73⁻ strain grown at 37° C. in LB medium and harvested at T2. The cDNA was generated using the specific primer vipR-GSP1. The subsequent PCR steps were realized with the nested gene-specific vipR-GSP2 and vipR-GSP3 primers. The P1 transcription start site of vipR was determined by sequencing the PCR product using the primer vipR-GSP3 and the P2 was identified by sequencing the PCR product using the primer midvipR-GSP3.

### Results

Expression of the vip3A Gene Requires the Presence of the Plasmid pBMB299.

To study the regulation of vip3A gene expression, the Inventors used the B. thuringiensis kurstaki HD73 strain as a heterologous and naive host which does not carry naturally the vip3A gene (22). Specifically, they have used a kurstaki strain designated HD73⁻ which was cured of the plasmid pHT73 carrying the cry1Ac gene (23). The transcriptional activity of the DNA fragment located upstream from the vip3A gene present on the pBMB299 plasmid (NZ_CP004876.1) of the B. thuringiensis kurstaki HD1 strain was analyzed. This DNA fragment of 709 bp was fused to the lacZ gene in the pHT304.18Z (24) and the resulting plasmid pHT-Pvip3 (FIG. 1A) was transformed into the HD73⁻ strain. HD73⁻ (pHT-Pvip3) bacteria were isolated on LB plates containing 50 μg/ml X-gal and the plates were observed after 24 h of growth at 37° C. No blue colonies were observed, indicating that the bacterial cells did not produce β-galactosidase activity (FIG. 1B). This result suggested that a specific regulator required for vip3A expression was absent from the HD73⁻ strain while it was present in the parental HD1 strain. The Inventors hypothesized that this regulator was encoded by a gene of the pBMB299. To demonstrate this hypothesis, they transferred the plasmid into the HD73⁻ SmR strain. In silico analysis of genes encoded on the pBMB299 revealed that a putative conjugal transfer protein (TraG) was present and suggested that this plasmid was conjugative. They introduced the pHT1618K into the HD1 strain by electroporation to select the conjugation event and thus increase the probability to find a clone carrying the pBMB299 among the HD73⁻ SmR KmR exconjugants that received the pHT1618K by a mobilization process. Indeed, it has been shown previously that the Gram-positive pBC16 replicon constituting pHT1618 is mobilizable by conjugation between B. thuringiensis strains (25). SmR KmR exconjugant clones having received the pHT1618K were screened for the presence of the pBMB299 by PCR using the primer pair vip3-fw/vip3-rev targeting the vip3A gene. 14% of these exconjugants harbored the pBMB299 and one clone HD73⁻ SmR KmR (pBMB299, pHT1618K) was selected. In agreement with the transfer of the pBMB299, the exconjugant clone produced bipyramidal crystals when grown on HCT plates for 4 days at 30° C. (FIG. 2). The strain was then cured of the pHT1618K by cultivating the bacteria on HCT plates until sporulation as previously described (26). Spores were then plated on LB plates and a SmR KmS colony having lost the pHT1618K was selected and the presence of the pBMB299 was confirmed by PCR as above. The Inventors then analyzed the production of the Vip3A protein by Western blot using an anti-Vip3A11 antibody. The HD73− SmR (pBMB299) and the HD73⁻ strains were cultivated in LB at 37° C., and bacterial cells and culture supernatants were collected at T1 and T4. The anti-Vip3A antibody did not reveal Vip3A in samples produced from the HD73⁻ SmR strain (FIG. 3). However, the Vip3A protein was detected in both the supernatant and the cell protein extract of the HD73⁻ SmR (pBMB299) strain. These results indicate that the HD73 strain is able to produce and secrete the toxin when it carries the pBMB299 and that the plasmid itself is able to specify the synthesis of Vip3A in the strain HD73. They therefore suggest that a regulator is encoded by a gene present on the pBMB299.

Identification of a Gene Involved in vip3A Expression.

To identify the pBMB299 gene(s) involved in vip3A expression, the Inventors performed a bioinformatic analysis on the vip3A-bearing plasmids from different B. thuringiensis strains (HD1, BGSC4C1, CT-43, HD12, HD29, IS5056, L7601, YBT-1520, YC-10). All of those are large plasmids with sizes ranging from 267 to 299 kb. In the HD1 strain, vip3A is located in a genetic environment containing multiple insertion sequences or transposase pseudogenes. Notably, vip3A is surrounded by two truncated DNA sequences showing similarities with the tnpB gene of the IS1341 family. An IS232 mobile element and a gene encoding a protein containing an N-terminal helix-turn-helix (HTH) domain putatively involved in DNA binding and annotated as a transcriptional antiterminator, are located upstream from vip3A (FIG. 4A). The orf-HTH gene is present at the same location in all the nine B. thuringiensis strains studied and the gene sequences displayed 90-100% of identity between strains (not shown). This gene was previously identified through a genomic analysis of the B. thuringiensis strain YBT1520 (15). The structural prediction of the protein encoded by the orf-HTH gene using the Phyre2 software (27) and an HHpred analysis revealed similarities to the Bacillus anthracis virulence regulator AtxA and the Streptococcus pneumonia virulence regulator Mga despite a low percentage of sequence identity (17 and 19%, respectively). The presence of this HTH-containing protein gene in the vicinity of vip3A in all strains led us to investigate its role in the activation of the transcription of the vip3A promoter (Pvip3). They constructed different transcriptional fusions with lacZ using DNA fragments of different lengths containing the Pvip3 and extending upstream to the end of the IS232 ATPase coding sequence as schematized in FIG. 4A. The plasmids pHT-Pvip3med1, pHT-Pvip3med2 and pHT-Pvip3long carrying these transcriptional fusions were transformed in strain HD73−. The resulting strains were plated on LB plates containing X-gal (FIG. 1C). The β-galactosidase activity (blue color) was only detected in the colonies of the HD73⁻ (pHT-Pvip3long) strain, suggesting that the DNA fragment containing the orf-HTH coding sequence was required for producing β-galactosidase. These results were confirmed by determining the expression kinetics of the 4 transcriptional fusions Pvip3-lacZ, Pvip3med1-lacZ, Pvip3med2-lacZ and Pvip3long-lacZ (FIG. 4B). The strains HD73− (pHT-Pvip3), HD73⁻ (pHT-Pvip3med1) and HD73⁻ (pHT-Pvip3med2) produced a very low amount of β-galactosidase throughout the growth of the bacteria (FIG. 4B). On the contrary, the β-galactosidase activity of the strain carrying the pHT-Pvip3long was high during the vegetative growth and increased significantly 1 h after the onset of the stationary phase. These results suggest that the orf-HTH gene is involved in the expression of the vip3A gene.

Analysis of the vip3A Promoter Region.

The Inventors determined the transcription start site (TSS) of the vip3A gene using total RNA samples from HD1 cells harvested at T2. The 5′ end of the vip3A transcript (designated as the putative TSS) was identified 403 bp upstream from the vip3A start codon (FIG. 5). This start is preceded by a potential −10 box (TATAAT) but no canonical SigA −35 box could be identified. Instead, analysis of the Pvip3 DNA sequence using the mFold software (28) found a palindromic DNA sequence having the potential to form a stem loop structure ending 29 bp upstream of the TSS (FIG. 5). These data and the β-galactosidase activity obtained with the Pvip3long-lacZ transcriptional fusion (FIG. 4B) reinforced the hypothesis that the protein encoded by the orf-HTH gene is the activator of the Pvip3 promoter through its binding to the stem-loop structure.

Characterization of the of the Regulator of vip3A Expression.

The structure prediction of the protein encoded by the orf-HTH gene indicated a similarity with Mga, the virulence regulator of S. pneumonia. The alanine replacement of two amino acids in the HTH domain of Mga abolished its DNA-binding activity and regulation of its target genes (29). To demonstrate that the protein (SEQ ID No45) encoded by the orf-HTH gene (SEQ ID No44) is responsible for the activation of the vip3A promoter, its HTH domain was modified based on its alignment with the HTH domain of the protein Mga (FIG. 6). Two residues of the potential HTH domain, W113 and S114 were replaced by two alanines (FIG. 7A) (SEQ ID No46 and 47). A transcriptional fusion between the Pvip3long fragment containing the mutations and the lacZ gene was constructed in the pHT304.18Z plasmid and the resulting pHT-Pvip3longmut plasmid was introduced into the HD73⁻ strain to generate the HD73⁻ (pHT-Pvip3long mut) strain. Comparison of the β-galactosidase activity of the HD73⁻ (pHT-Pvip3long-mut) and HD73⁻ (pHT-Pvip3long) strains showed that the mutation of the two amino acids completely abolished the transcriptional activity of the Pvip3long fragment (FIG. 7B). This result suggests that the protein encoded by the orf-HTH gene is involved in the activation of the Pvip3A promoter during early stationary phase. This transcriptional activator was named VipR for Vip Regulator. To confirm the activation of vip3A expression by VipR and to demonstrate that the lacZ transcription produced with the Pvip3long-lacZ transcriptional fusion originated from the promoter Pvip3, they introduced the pHT-Pvip3 in the HD73⁻ that harbors the pPxyl-vipR plasmid. In this strain, the expression of vipR was directed from the xylose inducible promoter Pxyl (FIG. 8A). The resulting HD73⁻ (pHT-Pvip3, pPxyl-vipR) strain was cultivated in LB in the presence or absence of xylose in the culture and the β-galactosidase activity of the cells was measured throughout the growth from T-1 to T7. They observed that the addition of xylose in the culture induced a strong increase in β-galactosidase production in the stationary phase (FIG. 8B). This demonstrated that the production of VipR activated lacZ transcription from the Pvip3. To determine whether the palindromic sequence located upstream of the vip3A TSS (FIG. 5) was involved in the control of vip3A expression, mutations were introduced to prevent this DNA sequence to form a stem-loop structure (FIG. 5C). A transcriptional fusion between the mutated Pvip3 promoter and lacZ was created in the pHT304.18Z and the resulting plasmid pHT-Pvip3-mut was transformed in the strain HD73− (pPxyl-vipR). Measurement of the β-galactosidase activity of the strains HD73⁻ (pPxyl-vipR, pHT-Pvip3) and HD73⁻ (pPxyl-vipR, pHT-Pvip3-mut) showed that the transcriptional activity of the mutated promoter was drastically lower than that of the wild-type promoter (FIG. 8D). This indicates that the palindromic sequence is required for full activation of vip3A transcription by VipR.

The vipR Gene is Autoregulated.

The expression kinetics of vipR was studied using a 1581 bp DNA fragment (PvipR) including the end of the IS232 and the first 355 bp of the vipR coding sequences (FIG. 9A). This DNA fragment was transcriptionally fused with lacZ in the pHT304.18Z plasmid and the resulting pHT-PvipR plasmid was transferred into the HD73⁻ strain. β-galactosidase activity of strain HD73⁻ (pHT-PvipR) was measured throughout the growth of bacteria cultivated in LB medium (FIG. 9B). Results showed that PvipR expression was low during the exponential phase of growth and increased 4-fold from T0 to T3. To determine if vipR expression is autoregulated, the Inventors introduced the plasmid pHT-PvipR in the strains HD73⁻ (pPxyl) and HD73⁻ (pPxyl-vipR) and compared the β-galactosidase activity of the two strains (FIG. 9C). The results showed that vipR transcription is significantly increased when VipR is produced in the bacterial cells under the control of Pxyl. Therefore, VipR presents the characteristics of an autoregulated transcriptional activator. They then determined the TSS of the vipR gene using total RNA samples from HD73− (pHT-Pvip3long) bacteria harvested at T2. A proximal vipR TSS named P1 was identified 575 bp upstream from the vipR start codon (FIG. 9D). This start is preceded by a potential −10 box (TATCTT). As for vip3A, no canonical SigA −35 box could be identified but a 16 bp palindromic sequence is present 32 bp upstream of this TSS. Alignment of this sequence with the palindrome sequence of the vip3A promoter allowed us to identify a conserved DNA sequence that might be the VipR binding site (FIG. 9E). This suggests that P1 is the autoregulated vipR promoter. The 5′-RACE PCR analysis of the vipR promoter indicated another TSS 998 bp upstream of the vipR start codon. This distal TSS, named P2, is preceded by a canonical −10 box (TAAAGT). A putative SigA −35 box (TGTAAAA) is correctly positioned 17 bp upstream of the −10 box suggesting that vipR transcription from the P2 promoter is SigA dependent. Identification of the two vipR promoters confirmed the results obtained with a Northern blot analysis of vipR transcription showing the detection of two transcripts in RNA samples of the HD73⁻ (pHT-Pvip3long) strain (FIG. 10). Discrete bands corresponding to the two transcripts were not detected in the HD1 and HD73⁻ (pBMB299) RNA samples. However, a light smear at the same place was present. The higher abundancy of the vipR transcripts in the HD73⁻ (pHT-Pvip3long) strain may be due to a higher copy number of the pHT-Pvip3long compared to the pBMB299.

### Identification of Putative VipR-Regulated Genes on Plasmids of the HD1 and HD73 Strains.

The Inventors searched for DNA sequences showing similarities with the putative VipR binding site in the plasmids pBMB299, pBMB65 and pBMB95 of the HD1 strain and in the plasmid pHT73 of the HD73 strain and identified a total of 10 conserved sequences located in the 5′ untranslated region of coding sequences (FIG. 11). The relevance of these sequences as potential VipR binding sites is greatly reinforced by the presence of a putative SigA −10 box 17 bp downstream from the last nucleotide of the consensus sequence. This result strongly suggests that the transcription of these 10 genes is at least partly controlled by VipR. In addition to vip3A and vipR, it appears that the expression of the cry1I gene expression might also be controlled by VipR. This result would be consistent with the observation that Cry1I toxin (formerly CryV) was produced in early stationary phase and exported, like Vip3A (30). More surprisingly, the two cry2A genes (formerly cryB or cry1I) are also located downstream from a putative VipR box. These genes are known to be transcribed by sporulation-specific sigma factors (31, 32), and a VipR-dependent expression would mean that the Cry2A toxins are also produced prior to the sporulation process, and thus prior to the formation of the parasporal crystal inclusion. Finally, five genes encoding N-acetylmuramoyl-L-alanine amidases (designated Amidase 1, 2, Amidase_{pBMB65}, Amidase_{pBMB95} and Amidase_{pHT73} are also located downstream of a putative VipR box (FIG. 11). The involvement of VipR in the activation of 3 promoters was studied using lacZ transcriptional fusions. A 381 bp DNA fragment upstream from the cry1I gene of the pBMB299, a 199 bp DNA fragment upstream from the amidase gene present on the pBMB95 plasmid and a 343 pb DNA fragment upstream from the amidase gene on the pHT73 plasmid were transcriptionally fused with lacZ in the pHT304.18Z and the resulting plasmids were transferred into HD73⁻ (pPxyl) and HD73⁻ (pPxyl-vipR) strains. Strains were patched on LB plates containing 50 μg/ml X-gal or HCT-glucose plates containing 100 μg/ml X-gal and 20 mM xylose and the plates were observed after 24 h of growth at 37° C. The β-galactosidase activity (blue color) was only detected in the colonies of the HD73⁻ strains harboring the pPxyl-vipR plasmid and therefore only when vipR is expressed in the cells (FIG. 13). This indicates that VipR is required for the expression of the cry1I gene and the two amidase genes.

vip3A and vipR Expression is Strongly Increased in a Δspo0A Genetic Background.

The activation of vipR and vip3A expression at the onset of stationary phase led the Inventors to address the role of a key regulator of the transition phase in Bacilli, Spo0A (33). vipR expression in the spo0A mutant was studied using a 2644-bp DNA fragment that includes the 5′ untranslated region upstream from vipR and the vipR coding sequence. This DNA fragment was cloned upstream of the lacZ gene in pHT304.18Z and the resulting plasmid, pHT-PvipR-vipR (FIG. 12A), was introduced in the HD73⁻ and HD73⁻ Δspo0A strains. vipR transcription was compared in these two genetic backgrounds. Levels of β-galactosidase produced by the bacteria indicated that, in the presence of vipR, a 40-fold increase in vipR transcription was observed in the Δspo0A mutant (FIG. 12B). To determine whether the vipR transcriptional increase resulted in an increase in vip3A expression, the Inventors measured the Pvip3A transcriptional activity in the HD73− Δspo0A mutant. In the Pvip3long DNA sequence, the vipR and vip3A orfs are in the same direction and no terminator sequence has been identified downstream vipR. They therefore cannot rule out the possibility that vipR transcription generates a polycistronic mRNA that includes vip3A. Thus, in order to disconnect vipR transcription from Pvip3A transcriptional activity, they constructed a plasmid carrying a vipR expression cassette oriented in the opposite direction compared to the Pvip3A-lacZ transcriptional fusion. A 2642-bp DNA fragment that includes the 5′ untranslated region upstream from vipR and the vipR coding sequence was cloned in the reverse orientation upstream of the Pvip3 DNA fragment in the pHT304.18Z plasmid (FIG. 12A). The resulting plasmid pHT-PvipR-vipR-Pvip3 was introduced in the HD73⁻ and HD73⁻ Δspo0A strains and the β-galactosidase activity of the two strains was compared (FIG. 12C). They observed that vip3 expression was strongly increased in the HD73⁻ Δspo0A strain compared to the HD73⁻ strain. Bioinformatic analysis of the DNA sequence upstream of the vipR coding sequence did not allow us to identify a DNA sequence corresponding to the B. subtilis Spo0A box (34). However, to determine if Spo0A controls vipR expression at a transcriptional level, the pHT-PvipR plasmid was introduced in the HD73⁻ Δspo0A strain and the β-galactosidase activity of the strain was compared to the activity of the HD73⁻ (pHT-PvipR) cells. The results showed that, in the absence of VipR, vipR transcription is similar in the two strains (FIG. 12D), indicating that Spo0A does not affect the transcriptional activity from the distal promoter P2.

Sequences listing:
SEQ ID Nº 1: vipR gene
ATGGATAAATTTATTACAGACTTAATACAAGATAAATCAATAGTA
CGACAACTTCAAATTTTAGAAACCTTGATTGATAGCAATGAAATA
AAGTCTTCCAAGGATTTATCACAAAGTTTAAAGTGTACAAGTAGA
ACAATAATAAATGACATTTCTCAGTTAAAACTAGCGCTTCCCGAA
AATTGGGATTTAATAAGTGTTCAATCCAAAGGGTACCTATTAAAA
CGTGATTTTTCAAACGATTTCTCCGAATTAATTATTCCCTATTTA
ATGAATAGTGAACTTTATACAATATTAATCGGTATATTTAATCAA
AAATATTATTCTTTAGAAAAATGGTCCCAGTTGTTATATCTAGAT
AAAATTACACTAAAAAAAATGCTGAAAAACTTTCGGAAGATACTT
GTGAACTTTGGATTGGATTTTAATTTTAGAACGATTAAATTGATT
GGCCAAGAAATAAACTTAAGATATTTTTATATAATGTTCTTTTAT
AATATCCAAAAGTATAAAGAAGTAATCAATTTAGATTCTAGATTA
CAAGAAAAAATTAAAAGCATTACTAGAATTCACAATGTAGAAATA
GATTATAATATGCTAGCAGTTATTATTAGTGTATCTATAAAGAGA
ATTGCAAATAAACATTATATGTCAGAAGTTTTAGAGTTTCTTCCT
ATACTTGATACAAATAATTTAAATTGCATAAGTTCAATTATATGC
GAATTGGAAATTTTCTTCAATATAAAATTCACAGAATATGAATTA
AGTTATTTTAAGAATGCTTTTTCAATAATATTAGAATGGAACATT
GAAGAAAAAAATAGGATCACAGATTATTATTATAAAATAAACAAA
AAGACTTATGATAAAAACAAACATTTATTCCAAACAATATCTTCT
GAAATAAATGTTTGTTTAGAAATAAAAGAAAAGATAAAACATGAC
TTGTATTTCATTTTACATAAAATTTACAAGTTTCAAAAGTATGGT
TTATCAATAGGCGCTTTTGGTAATGAATTTGACACTGTACATCAT
GAATTTTCAGAAGGACATAATAGAATTTATCCTCTTATTTCTTCT
TGGAATAAAAAAATAAATAAAAGTAGATTAACTAACGATGAGATC
AATTACATAATATACCATATTTTATTTATTGTCCATTCAAATCAT
AATAAAAAAGGATTGTTATTATTATCTGGTTCATCAGCTTTAAAG
AAGTTTATTTATTATAAACTCAATCACGAGCTAGGTGATTTTGTA
ACACTACAACAAAAACCAGATTGTATGCATAAATTTGATGTTATT
CTGACAAATTATCAAATCCCGAATACCCCAATACCTATAATTCGG
ATCTCAAACAAAACAATTCAAAAAGATTCAAGTTATGTTAGAAAA
GTTTTATCCCCTTTTAACTAA
SEQ ID Nº 2: VipR protein
MDKFITDLIQDKSIVRQLQILETLIDSNEIKSSKDLSQSLKCTSR
TIINDISQLKLALPENWDLISVQSKGYLLKRDFSNDFSELIIPYL
MNSELYTILIGIFNQKYYSLEKWSQLLYLDKITLKKMLKNFRKIL
VNFGLDFNFRTIKLIGQEINLRYFYIMFFYNIQKYKEVINLDSRL
QEKIKSITRIHNVEIDYNMLAVIISVSIKRIANKHYMSEVLEFLP
ILDTNNLNCISSIICELEIFFNIKFTEYELSYFKNAFSILEWNIE
EKNRITDYYYKINKKTYDKNKHLFQTISSEINVCLEIKEKIKHDL
YFILHKIYKFQKYGLSIGAFGNEFDTVHHEFSEGHNRIYPLISSW
NKKINKSRLTNDEINYIIYHILFIVHSNHNKKGLLLLSGSSALKK
FIYYKLNHELGDFVTLQQKPDCMHKFDVILTNYQIPNTPIPIIRI
SNKTIQKDSSYVRKVLSPFN
SEQ ID Nº 3: promoter Pvip3 (intergenic sequence)
GACTGTCCTTCTTATCTTACACGCTATAATGCGGATATTAACTCA
ATAAGCCATAAGGCTTCTAATCGCAAGGACTGCTGGGGTAGTTCA
ATAGCTCTGGTATAAATAGCAGTAAAATAACCTTTGGTACAATTC
GATAGTTACTAGACGTAACGATTAGCCGAGAAATCCCCACTTTAG
AGAGCACGTTAGGAGGTTAAGTGAAGGGTATTTCACAAATGATCG
ATTATATTTATAGGAAAAGGATAGATCACTTCATCTATAGATGAA
ATTATCTATCCTTTTATTTGTTAGATAGGAGATATAATTTACTCA
TAGCCCCAGAGAAAGTGTTAACAGGTGATAGCATCCAATTTTATA
GTAAGGGAGGTTTTACTAAGTTCTAATAATTTTCTGGGTAATTAG
TCCTTACAACGACTTACATTTAGGAAATTGCCTGTTAATAAAATT
TATATAGTAAAGGAGTGCTTTAATGTAAAAAAATAGGGATAAATG
AATTTTTGTGAATAGGAAGACCATTTGATAGGTAAATATTATAAT
TCAAAAAGTAGAATAAGCAAAATTGAGTAACACATTAATGATATA
AACTAATTTTATACAATTGAAATTGATAAAAAGTTATGAGTGTTT
AATAATCAGTAATTACCAATAAAGAATTAAGAATACAAGTTTACA
AGAAATAAGTGTTACAAAAAATAGCTGAAAAGGAAGATGAAC
SEQ ID Nº 4: promoters consensus sequence
TTC-N-N-N-N-AT-N-G-N-N-GAA-N-N-TAT-N-T-N-T-
N-CTTT
wherein N is A or T or C or G
SEQ ID Nº 5: promoter PvipR
TTTTGTACATGCTTAAACAAGCGAAAATGTACATGTTTATCTTGA
CATTTACAAAGAGATGAATATATTTGAATTTTATTAATTAAATCC
AGAAAAAAATATCAGAATAGAAAATGCTTTAAGGCCTATTTGCAG
TTGAATATTTATGGTATGAATATTGCCGTATTTCTTAGTATGCTA
AAAATACAAGCCTGTAAATCACTTTGTTATGACTGTAAAGTAAAA
GAAGGATATTGAACTATTTTATTATTTATAAGGTATTATATATAC
TTTGAGATTCAGAAGGTTTGAATAAGTTACGCTTTATATGCCGAT
ATTTTGGTTCAACTAGAGTATCAAATGTGGAATATTTCTTTGCTA
TATAAAACCTTTACTTCTTAATCTTATATAAGCACAAAGTAAAAC
ATAAGGGGATTTTTGAAAAAAGAGTATTATGGTCTAGTATAAAAC
TTGTATTTTAGAACTTTAAATATATTAAGATAATTCAAAGGGGAA
AATAAAATCCAATAATCATTAAGATGGTAGTTTCTTTGGATTCGT
TTCTTAAAATATCTATTTTTGATTTTCCTTATCTATTTTGTAATA
AAACTTTTCATTAATAGATGAAAATATGTATGCTTTATTAGGAAA
TAATTAAGGTATCTTTTAATTATCTATAATTATTTGTTATAGAGG
AATTAGATAATTGAAGTTCTTTTCATTTGGGAATACGGTGATAAA
AAAGGAGGGACGGAGTTATAAGGATCAATAAGAATACATAATTAA
ATTGTTTTACAATAAAACATTCCATTCTCATTAATATTCTTATAA
TCACAAAAACCAAATGCTATAAAATGGATTGTTTGAAAAAGTTGT
GAATATGCATATTTTTGGATGCGTGTACAATTATAATCATCATGA
TATCACTTCTCTTTTAAGAATGCGAAGTTTTGTCGTTATGTAAAC
ATGGGTGCTCAAAACTTTACATTTTTTGTACAATATAAAATATTA
AAATACAGTTGTACAGATAATTGATATTTCTTTATATGAAAAAAT
GCTTATAAATACTATGCAAAGCTAACGTATAATTTTTTAGACCTT
ATAGAATCAAATAAGAATAAAGATAGAAAATAAAATTGAAATACT
AAGGCTAGCAAAGTAACGTGAGGTAATAGCATCTATTCTGAGTTT
TGTAGTTAAATGTTACCTAGTGATAAGTAATTCAGTATTAAGGGA
GGAGAAATGTA
SEQ ID Nº 6: promoter Pamidase 1
CCTAAAATTTCAAGGCATTTTAAATGTTTTGTCTAGAAAAGTATG
GATAACTTCATGAATGGGTGAAAATATCTATGCTTTTTTATTACA
AGACAAAGATATACTGTACTCATAA
SEQ ID Nº 7: promoter Pamidase 2
GCTTTTAAGAACCCCTATTTTAAATGTITTGTCTAGAAAAGTATG
GATAACTTCATGAATGGGTGAAAATATCTATGCTTTTTTATTACA
AGACAAAGATATACTGTACTCATAA
SEQ ID N°8: promoter Pcry1l
TATTTTCTAGTAATACATATGTATAGAGCAACTTAATCAAGCAGA
GATATTTTCACCTATCGATGAAAATATCTCTGCTTTTTCTTTTTT
TATTTGGTATATGCTTTACTTGTAA
SEQ ID Nº 9: promoter Pcry2Ab
CACTTCTAAATGAAGTGAAAGTGGGGGTAGTTCAAAAAAAGCATA
GATATCTTCTTCTATAGGTGAAGATATCTATGCTTTTTCTTTTTA
AATTAAAGATATACTTTACTCATAC
SEQ ID Nº 10: Vip3A protein
MNKNNTKLSTRALPSFIDYFNGIYGFATGIKDIMNMIFKTDTGGD
LTLDEILKNQQLLNDISGKLDGVNGSLNDLIAQGNLNTELSKEIL
KIANEQNQVLNDVNNKLDAINTMLRVYLPKITSMLSDVMKQNYAL
SLQIEYLSKQLQEISDKLDIINVNVLINSTLTEITPAYQRIKYVN
EKFEELTFATETSSKVKKDGSPADILDELTELTELAKSVTKNDVD
GFEFYLNTFHDVMVGNNLFGRSALKTASELITKENVKTSGSEVGN
VYNFLIVLTALQAKAFLTLTTCRKLLGLADIDYTSIMNEHLNKEK
EEFRVNILPTLSNTFSNPNYAKVKGSDEDAKMIVEAKPGHALIGF
EISNDSITVLKVYEAKLKQNYQVDKDSLSEVIYGDMDKLLCPDQS
EQIYYTNNIVFPNEYVITKIDFTKKMKTLRYEVTANFYDSSTGEI
DLNKKKVESSEAEYRTLSANDDGVYMPLGVISETFLTPINGFGLQ
ADENSRLITLTCKSYLRELLLATDLSNKETKLIVPPSGFISNIVE
NGSIEEDNLEPWKANNKNAYVDHTGGVNGTKALYVHKDGGISQFI
GDKLKPKTEYVIQYTVKGKPSIHLKDENTGYIHYEDTNNNLEDYQ
TINKRFTTGTDLKGVYLILKSQNGDEAWGDNFIILEISPSEKLLS
PELINTNNWTSTGSTNISGNTLTLYQGGRGILKQNLQLDSFSTYR
VYFSVSGDANVRIRNSREVLFEKRYMSGAKDVSEMFTTKFEKDNF
YIELSQGNNLYGGPIVHFYDVSIK
SEQ ID Nº 11: cry1l protein
MKLKNQDKHQSFSSNAKVDKISTDSLKNETDIELQNINHEDCLKM
SEYENVEPFVSASTIQTGIGIAGKILGTLGVPFAGQVASLYSFIL
GELWPKGKNQWEIFMEHVEEIINQKISTYARNKALTDLKGLGDAL
AVYHDSLESWVGNRNNTRARSVVKSQYIALELMFVQKLPSFAVSG
EEVPLLPIYAQAANLHLLLLRDASIFGKEWGLSSSEISTFYNRQV
ERAGDYSDHCVKWYSTGLNNLRGTNAESWVRYNQFRRDMTLMVLD
LVALFPSYDTQMYPIKTTAQLTREVYTDAIGTVHPHPSFTSTTWY
NNNAPSFSAIEAAVVRNPHLLDFLEQVTIYSLLSRWSNTQYMNMW
GGHKLEFRTIGGTLNISTQGSTNTSINPVTLPFTSRDVYRTESLA
GLNLFLTQPVNGVPRVDFHWKFVTHPIASDNFYYPGYAGIGTQLQ
DSENELPPEATGQPNYESYSHRLSHIGLISASHVKALVYSWTHRS
ADRTNTIEPNSITQIPLVKAFNLSSGAAVVRGPGFTGGDILRRTN
TGTFGDIRVNINPPFAQRYRVRIRYASTTDLQFHTSINGKAINQG
NFSATMNRGEDLDYKTFRTVGFTTPFSFLDVQSTFTIGAWNFSSG
NEVYIDRIEFVPVEVTYEAEYDFEKAQEKVTALFTSTNPRGLKTD
VKDYHIDQVSNLVESLSDEFYLDEKRELFEIVKYAKQLHIERNM
SEQ ID Nº 12: cry2Ab protein
MNSVLNSGRTTICDAYNVAAHDPFSFQHKSLDTVQKEWTEWKKNN
HSLYLDPIVGTVASFLLKKVGSLVGKRILSELRNLIFPSGSTNLM
QDILRETEKFLNQRLNTDTLARVNAELTGLQANVEEFNRQVDNFL
NPNRNAVPLSITSSVNTMQQLFLNRLPQFQIQGYQLLLLPLFAQA
ANLHLSFIRDVILNADEWGISAATLRTYRDYLKNYTRDYSNYCIN
TYQSAFKGLNTRLHDMLEFRTYMFLNVFEYVSIWSLFKYQSLLVS
SGANLYASGSGPQQTQSFTSQDWPFLYSLFQVNSNYVLNGFSGAR
LSNTFPNIVGLPGSTTTHALLAARVNYSGGISSGDIGASPFNQNF
NCSTFLPPLLTPFVRSWLDSGSDREGVATVTNWQTESFETTLGLR
SGAFTARGNSNYFPDYFIRNISGVPLVVRNEDLRRPLHYNEIRNI
ASPSGTPGGARAYMVSVHNRKNNIHAVHENGSMIHLAPNDYTGFT
ISPIHATQVNNQTRTFISEKFGNQGDSLRFEQNNTTARYTLRGNG
NSYNLYLRVSSIGNSTIRVTINGRVYTATNVNTTTNNDGVNDNGA
RFSDINIGNVVASSNSDVPLDINVTLNSGTQFDLMNIMLVPTNIS
PLY
SEQ ID Nº 13: STAB SD
gaaaggaggg atgcc
SEQ ID Nº 14: Tcry
aaactcaggt ttaaatatcg ttttcaaatc aattgtccaa
gagcagcatt acaaatagat aagtaatttg ttgtaatgaa
aaacggacat cacctccatt gaaacggagt gatgtccgtt
ttactatgtt attttctagt aatacatatg tatagagcaa
cttaatcaag cagagatatt
SEQ ID Nº 15: promoter Pvip3
AAGGGTATTTCACAAATGATCGATTATATTTATAGGAAAAGGATA
GATCACTTCATCTATAGATGAAATTATCTATCCTTTTATTTGTTA
GATAGGAGATATAATTTACTCATAG
SEQ ID Nº 72: promoter PamidasePbmb65
GCTTTTAAGAACCCCTATTTTAAATGTTTTGTCTAGAAAAGTATG
GATAACTTCATGAATGGGTGAAAATATCTATGCTTTTTTATTACA
AGACAAAGATATACTGTACTCATAA
SEQ ID Nº 73: promoter PamidasepBMB65
(intergenic sequence)
ACTTAGGACATTGAACTACTCACCACTTAACGTCCTTACAGACTT
TTTGAAGTGGGAGATTCCTGCTAAGATAACCAATGTATCCACCGG
TTATCTTAGCAGGCGCTACCCGTAGTCCCTACAGTGAGAACATAA
GTATCTTGTACATTGACGCACCACACTCCCTACTTTAGTTTGGTT
TTCGCAACTTCGGCAAGCATGGGGCGAAAGAACGTTGAAGATTTT
ACAGTTGCAACGTTTTTCTGAAACCAACCTCATAACTTCAGTTTT
CGAAGAGCAATCTGTATAAGTAAATGATAGCAAATATGTTGGCTT
ATGCCTATCGATATTCATATCCCACCTAAAATTGGTGTTTCACAC
CTTGACATTTTTGAGGAAGGAGTCTTCTGTTGGAAAACGATAAAA
TCTTTTTAATTACTTGAATTCTAAGTGTAGAAACTTTGTAATTTG
TATGAATTGAGGGATTGAATTCCTTGTGTTAATTAACATATATAT
CGATTTGATATAAAGATAGGATCTAAAAGGAGCTAACAAACTGTT
AGCTCCTTTTAGATCCTTTTTAAGTATATCGCACAAAAATCAAAT
CATACATCACGAAAAGTAGCTTTTAAGAACCCCTATTTTAAATGT
TTTGTCTAGAAAAGTATGGATAACTTCATGAATGGGTGAAAATAT
CTATGCTTTTTTATTACAAGACAAAGATATACTGTACTCATAAAT
GTAGAAATATCTATTTAAGAAAGATTGAAATCATATCCAATAATC
TGATAGG
SEQ ID Nº 74: promoter Pamidase_{pBMB95}
CCTAAAATTTCAAGGCATTTTAGATGTTTTATGTGGAAAAGCATG
GTTAACTTCATCAATAGGTGAAAATATCTATGCTTTTTTATTTCA
GGACAGAGATATACTGTACTCATAA
SEQ ID Nº 65: promoter Pamidase_{pBMB95}
(intergenic sequence)
ATCACGAAAAGAAGTTTTTAAGAACCATTCCTAAAATTTCAAGGC
ATTTTAGATGTTTTATGTGGAAAAGCATGGTTAACTTCATCAATA
GGTGAAAATATCTATGCTTTTTTATTTCAGGACAGAGATATACTG
TACTCATAAATATAGAAATATGTATTTAAGAAAGATTGAAAGCGT
ATTAAATAATCTGATAGGA
SEQ ID Nº 75: promoter Pamidase_{pHT73}
GCTTTTAAGAACCCCTATTTTAAATGTTTTGTCTAGAAAAGTATG
GATAACTTCATGAATGGGTGAAAATATCTATGCTTTTTTATTACA
AGACAAAGATATACTGTACTCATAA
SEQ ID Nº 66: promoter Pamidase_{pHT73}
(intergenic sequence)
TGTAGAAACTTTGTAATTTGTATGAATTGAGGGATTGAATTCCTT
GTGTTAATTAACATATATATCGATTTGATATAAAGATAGGATCTA
AAAGGAGCTAACAAACTGTTAGCTCCTTTTAGATCCTTTTTAAGT
ATATCGCACAAAAATCAAATCATACATCACGAAAAGTAGCTTTTA
AGAACCCCTATTTTAAATGTTTTGTCTAGAAAAGTATGGATAACT
TCATGAATGGGTGAAAATATCTATGCTTTTTTATTACAAGACAAA
GATATACTGTACTCATAAATGTAGAAATATCTATTTAAGAAAGAT
TGAAATCATATCCAATAATCTGATAGGA
SEQ ID Nº 76: promoter Pcry1l
(intergenic sequence)
GTGATGTCCGTTTTACTATGTTATTTTCTAGTAATACATATGTAT
AGAGCAACTTAATCAAGCAGAGATATTTTCACCTATCGATGAAAA
TATCTCTGCTTTTTCTTTTTTTATTTGGTATATGCTTTACTTGTA
ATCGAAAATAAAGCACTAATAAGAGTATTTATAGGTGTTTGAAGT
TATTTCAGTTCATTTTTAAAGAAGGTTTAAAGACGTTAGAAAGTT
ATTAAGGAATAATATTTATTAGTAAATTCCACATATATTATATAA
TTAATTATGAAATATATGTATAAATTGAAAATGCTTTATTTGACA
TTACAGCTAAGTATAATTTTGTATGAATAAAATTATATCTGAAAA
TTAAATAATAGTATAAGTGGA

### REFERENCES

1. Ehling-Schulz M, Lereclus D, Koehler T M. 2019. The Bacillus cereus group: Bacillus species with pathogenic potential. Microbiol Spectr 7.
2. Schnepf E, Crickmore N, Van Rie J, Lereclus D, Baum J, Feitelson J, Zeigler D R, Dean D H. 1998. Bacillus thuringiensis and its pesticidal crystal proteins. Microbiol Mol Biol Rev 62:775-806.
3. Crickmore N, Berry C, Panneerselvam S, Mishra R, Connor T R, Bonning B C. 2021. A structure-based nomenclature for Bacillus thuringiensis and other bacteria derived pesticidal proteins. J Invertebr Pathol 186:107438.
4. Crickmore N, Zeigler D R, Feitelson J, Schnepf E, Van Rie J, Lereclus D, Baum J, Dean D H. 1998. Revision of the nomenclature for the Bacillus thuringiensis pesticidal crystal proteins. Microbiol Mol Biol Rev 62:807-13.
5. Deng C, Peng Q, Song F, Lereclus D. 2014. Regulation of cry gene expression in Bacillus thuringiensis. Toxins (Basel) 6:2194-209.
6. Lereclus D, Agaisse H, Gominet M, Chaufaux J. 1995. Overproduction of encapsulated insecticidal crystal proteins in a Bacillus thuringiensis spo0A mutant. Nat Biotechnol 13:67-71.
7. Zhang R, Slamti L, Tong L, Verplaetse E, Ma L, Lemy C, Peng Q, Guo S, Zhang J, Song F, Lereclus D. 2020. The stationary phase regulator CpcR activates cry gene expression in non-sporulating cells of Bacillus thuringiensis. Mol Microbiol 113:740-754.
8. van Frankenhuyzen K. 2009. Insecticidal activity of Bacillus thuringiensis crystal proteins. J Invertebr Pathol 101:1-16.
9. Raymond B, Johnston P R, Nielsen-LeRoux C, Lereclus 600 D, Crickmore N. 2010. Bacillus thuringiensis: an impotent pathogen? Trends Microbiol 18:189-194.
10. Salamitou S, Ramisse F, Brehelin M, Bourguet D, Gilois N, Gominet M, Hernandez E, Lereclus D. 2000. The plcR regulon is involved in the opportunistic properties of Bacillus thuringiensis and Bacillus cereus in mice and insects. Microbiology 146:2825-2832.
11. Donovan W P, Donovan J C, Engleman J T. 2001. Gene knockout demonstrates that vip3A contributes to the pathogenesis of Bacillus thuringiensis toward Agrotis ipsilon and Spodoptera exigua. J Invertebr Pathol 78:45-51.
12. Estruch J J, Warren G W, Mullins M A, Nye G J, Craig J A, Koziel M G. 1996. Vip3A, a novel Bacillus thuringiensis vegetative insecticidal protein with a wide spectrum of activities against lepidopteran insects. Proc Natl Acad Sci USA 93:5389-5394.
13. Espinasse S, Chaufaux J, Buisson C, Perchat S, Gohar M, Bourguet D, Sanchis V. 2003. Occurrence and linkage between secreted insecticidal toxins in natural isolates of Bacillus thuringiensis. Curr Microbiol 47:501-7.
14. Hernandez-Rodriguez C S, Boets A, Van Rie J, Ferre J. 2009. Screening and identification of vip genes in Bacillus thuringiensis strains. J Appl Microbiol 107:219-25.
15. Zhu L, Peng D, Wang Y, Ye W, Zheng J, Zhao C, Han D, Geng C, Ruan L, He J, Yu Z, Sun M. 2015.

Genomic and transcriptomic insights into the efficient entomopathogenicity of Bacillus thuringiensis. Sci Rep 5:14129.
16. Ruiz de Escudero I, Banyuls N, Bel Y, Maeztu M, Escriche B, Munoz D, Caballero P, Ferre J. 2014. A screening of five Bacillus thuringiensis Vip3A proteins for their activity against lepidopteran pests. J Invertebr Pathol 117:51-5.
17. Chakroun M, Banyuls N, Bel Y, Escriche B, Ferre J. 2016. 624 Bacterial Vegetative Insecticidal Proteins (Vip) from Entomopathogenic Bacteria. Microbiol Mol Biol Rev 80:329-50.
18. Gouffon C, Van Vliet A, Van Rie J, Jansens S, Jurat-Fuentes J L. 2011. Binding sites for Bacillus thuringiensis Cry2Ae toxin on heliothine brush border membrane vesicles are not shared with Cry1A, Cry1F, or Vip3A toxin. Appl Environ Microbiol 77:3182-8.
19. Liu J G, Yang A Z, Shen X H, Hua B G, Shi G L. 2011. Specific binding of activated Vip3Aa10 to Helicoverpa armigera brush border membrane vesicles results in pore formation. J Invertebr Pathol 108:92-7.
20. Sena J A, Hernandez-Rodriguez C S, Ferre J. 2009. Interaction of Bacillus thuringiensis Cry1 and Vip3A proteins with Spodoptera frugiperda midgut binding sites. Appl Environ Microbiol 75:2236-7.
21. Carriere Y, Crickmore N, Tabashnik B E. 2015. Optimizing pyramided transgenic Bt crops for sustainable pest management. Nat Biotechnol 33:161-8.
22. Liu G, Song L, Shu C, Wang P, Deng C, Peng Q, Lereclus D, Wang X, Huang D, Zhang J, Song F. 2013. Complete genome sequence of Bacillus thuringiensis subsp. kurstaki strain HD73. Genome Announc 1:e0008013.
23. Wilcks A, Jayaswal N, Lereclus D, Andrup L. 1998. Characterization of plasmid pAW63, a second self-transmissible plasmid in Bacillus thuringiensis subsp. kurstaki HD73. Microbiology 144 (Pt 5):1263-70.
24. Agaisse H, Lereclus D. 1994. Structural and functional analysis of the promoter region involved in full expression of the cryIIIA toxin gene of Bacillus thuringiensis. Mol Microbiol 13:97-107.
25. Lereclus D, Menou G, Lecadet M-M. 1983. Isolation of a DNA sequence related to several plasmids from Bacillus thuringiensis after a mating involving the Streptococcus faecalis plasmid pAMs1. Mol Gen Genet 191:307-313.
26. Lereclus D, Arantes O. 1992. spbA locus ensures the segregational stability of pHT1030, a novel type of Gram-positive replicon. Mol Microbiol 7:35-46.
27. Kelley L A, Mezulis S, Yates C M, Wass M N, Sternberg M J. 2015. The Phyre2 web portal for protein modeling, prediction and analysis. Nat Protoc 10:845-58.
28. Zuker M. 2003. Mfold web server for nucleic acid folding and hybridization prediction. Nucleic Acids Res 31:3406-15.
29. McIver K S, Myles R L. 2002. Two DNA-binding domains of Mga are required for virulence gene activation in the group A streptococcus. Mol Microbiol 43:1591-601.
30. Kostichka K, Warren G W, Mullins M, Mullins A D, Palekar N V, Craig J A, Koziel M G, Estruch J J. 1996. Cloning of a cryV-type insecticidal protein gene from Bacillus thuringiensis: the cryV-encoded protein is expressed early in stationary phase. J Bacteriol 178:2141-4.
31. Baum J A, Malvar T. 1995. Regulation of insecticidal crystal protein production in Bacillus thuringiensis. Mol Microbiol 18:1-12.
32. Widner W R, Whiteley H R. 1989. Two highly related insecticidal crystal proteins of Bacillus thuringiensis subsp. kurstaki possess different host range specificities. J Bacteriol 171:965-74.
33. Sonenshein A L. 2000. Control of sporulation initiation in Bacillus subtilis. Curr Opin Microbiol 3:561-6.
34. Molle V, Fujita M, Jensen S T, Eichenberger P, Gonzalez-Pastor J E, Liu J S, Losick R. 2003. The Spo0A regulon of Bacillus subtilis. Mol Microbiol 50:1683-701.
35. Wang Z, Gan C, Wang J, Bravo A, Soberon M, Yang Q, Zhang J. 2021. Nutrient conditions determine the localization of Bacillus thuringiensis Vip3Aa protein in the mother cell compartment. Microb Biotechnol 14:551-560.
36. Helmann J D, Chamberlin M J. 1988. Structure and function of bacterial sigma factors. Annu Rev Biochem 57:839-72.
37. Rhodius V A, Busby S J. 1998. Positive activation of gene expression. Curr Opin Microbiol 1:152-9.
38. Burbulys D, Trach K A, Hoch J A. 1991. Initiation of sporulation in B. subtilis is controlled by a multicomponent phosphorelay. Cell 64:545-552.
39. Fagerlund A, Dubois T, Okstad O A, Verplaetse E, Gilois N, Bennaceur I, Perchat S, Gominet M, Aymerich S, Kolsto A B, Lereclus D, Gohar M. 2014. SinR controls enterotoxin expression in Bacillus thuringiensis biofilms. PLoS One 9:e87532.
40. Sonenshein A L. 2005. CodY, a global regulator of stationary phase and virulence in Gram-positive bacteria. Curr Opin Microbiol 8:203-7.
41. Strauch M A. 1993. Regulation of Bacillus subtilis gene expression during the transition from exponential growth to stationary phase, p 121-153, Progress in nucleic acid research and molecular biology, Academic Press, Inc ed, vol 46.
42. Lereclus D, Agaisse H, Grandvalet C, Salamitou S, Gominet M. 2000. Regulation of toxin and virulence gene transcription in Bacillus thuringiensis. Int J Med Microbiol 290:295-299.
43. Strauch M, Webb V, Spiegelman G, Hoch J A. 1990. The Spo0A protein of Bacillus subtilis is a repressor of the abrB gene. Proc Natl Acad Sci USA 87:1801-1805.
44. Rom J S, Hart M T, McIver K S. 2021. PRD-Containing Virulence Regulators (PCVRs) in Pathogenic Bacteria. Front Cell Infect Microbiol 11:772874.
45. Hammerstrom T G, Horton L B, Swick M C, Joachimiak A, Osipiuk J, Koehler T M. 2015. Crystal structure of Bacillus anthracis virulence regulator AtxA and effects of phosphorylated histidines on multimerization and activity. Mol Microbiol 95:426-41.
46. Tsvetanova B, Wilson A C, Bongiorni C, Chiang C, Hoch J A, Perego M. 2007. Opposing effects of histidine phosphorylation regulate the AtxA virulence transcription factor in Bacillus anthracis. Mol Microbiol 63:644-55.
47. Hondorp E R, Hou S C, Hause L L, Gera K, Lee C E, McIver K S. 2013. PTS phosphorylation of Mga modulates regulon expression and virulence in the group A streptococcus. Mol Microbiol 88:1176-93.
48. Dubois N R, Dean D H. 1995. Synergism between Cry1A insecticidal crystal proteins and spores of Bacillus thuringiensis, other bacterial spores and vegetative cells against Lymantria dispar (Lepidoptera: Lymantriidae) larvae. Environ Entomol 24:1741-1747.
49. Yang J, Peng Q, Chen Z, Deng C, Shu C, Zhang J, Huang D, Song F. 2013. Transcriptional regulation and characteristics of a novel N-acetylmuramoyl-Lalanine amidase gene involved in Bacillus thuringiensis mother cell lysis. J Bacteriol 195:2887-97.
50. Wydau-Dematteis S, El Meouche I, Courtin P, Hamiot A, Lai-Kuen R, Saubamea B, Fenaille F, Butel M J, Pons J L, Dupuy B, Chapot-Chartier M P, Peltier J. 2018. Cwp19 Is a Novel Lytic Transglycosylase Involved in Stationary-Phase Autolysis Resulting in Toxin Release in Clostridium difficile. mBio 9.
51. MacNeil D J, Gewain K M, Ruby C L, Dezeny G, Gibbons P H, MacNeil T. 1992. Analysis of Streptomyces avermitilis genes required for avermectin biosynthesis utilizing a novel integration vector. Gene 111:61-8.
52. Lereclus D, Arantes O, Chaufaux J, Lecadet M-M. 1989. Transformation and expression of a cloned ∂-endotoxin gene in Bacillus thuringiensis. FEMS Microbiol Lett 60:211-218.
53. Macaluso A, Mettus A M. 1991. Efficient transformation of Bacillus thuringiensis requires nonmethylated plasmid DNA. J Bacteriol 173:1353-6.
54. Soding J, Biegert A, Lupas A N. 2005. The HHpred interactive server for protein homology detection and structure prediction. Nucleic Acids Res 33:W244-8.
55. Bradford M M. 1976. A rapid and sensitive method for the quantitation of microgram quantities of protein utilizing the principle of protein-dye binding. Anal Biochem 72:248-54.
56. Wang Z, Fang L, Zhou Z, Pacheco S, Gomez I, Song F, Soberon M, Zhang J, Bravo A. 2018. Specific binding between Bacillus thuringiensis Cry9Aa and Vip3Aa toxins synergizes their toxicity against Asiatic rice borer (Chilo suppressalis). J Biol Chem 293:11447-11458.
57. Perchat S, Dubois T, Zouhir S, Gominet M, Poncet S, Lemy C, Aumont-Nicaise M, Deutscher J, Gohar M, Nessler S, Lereclus D. 2011. A cell-cell communication system regulates protease production during sporulation in bacteria of the Bacillus cereus group. Mol Microbiol 82:619-33.

1. A vipR regulator gene comprising at least 90% identity, at least 95% identity, at least 98% identity, or 100% identity to the polynucleotide sequence of SEQ ID NO:1, wherein the vipR regulator gene is configured to direct expression of at least one promoter that comprises the polynucleotide sequence of SEQ ID NO:4,
wherein the at least one promoter is positioned at least 15 to 21 nucleotides upstream of a Sigma A-specific −10 box that precedes a start codon of a gene encoding a protein of interest.

2. The vipR regulator gene of claim 1, wherein the at least one promoter is selected from the group consisting of: PvipR SEQ ID NO:5), Pvip3 SEQ ID NO:15 or SEQ ID NO:3), Pamidase-1 SEQ ID NO:6), Pamidase-2 SEQ ID NO:7), -Pcry1I SEQ ID NO:8 or SEQ ID NO:76), Pcry2Ab SEQ ID NO:9) Pamidase_{pBMB65} SEQ ID NO:72 or SEQ ID NO:73), Pamidase_{pBMB95} SEQ ID NO:65 or SEQ ID NO:74) and Pamidase_{pHT73} SEQ ID NO:66 or SEQ ID NO:75); or wherein the at least one promoter is Pvip3 SEQ ID NO:3) and Pcry1I SEQ ID NO:8).

3. The vipR regulator gene of claim 1, wherein the protein of interest is selected from the group consisting of: Vip3A, and Cry1I.

4. An expression system configured for expression of a protein of interest in a Bacillus strain, the expression system comprising:
at least one gene comprising at least one polynucleotide sequence encoding the protein of interest; and
at least one first promoter comprising the polynucleotide sequence of SEQ ID NO:4,
wherein the polynucleotide sequence of SEQ ID NO:4 of the at least one first promoter is positioned at least 14 nucleotides upstream of a Sigma A-specific −10 box of the expression system that precedes a start codon of the at least one gene; and
a vipR regulator gene comprising at least 90% identity, at least 95% identity, at least 98% identity, or 100% identity to the polynucleotide sequence of SEQ ID NO:1, wherein the vipR regulator gene is configured to direct expression of the at least one first promoter, and wherein expression of the vipR regulator gene is directed by a second promoter that comprises the polynucleotide sequence of SEQ ID NO:5.

5. The expression system of claim 4, wherein the at least one first promoter is Pvip3 and comprises the polynucleotide sequence of SEQ ID NO:3.

6. The expression system of claim 4, wherein the at least one protein of interest is selected from the group consisting of: Vip3A, and Cry1I.

7. The expression system of claim 4, wherein the expression system comprises a stabilizing sequence of an mRNA of a cry1Ac gene, a terminator sequence of the cry1Ac gene, or both.

8. The expression system of claim 4, wherein the expression system is comprised into at least one vector.

9. The expression system of claim 8, wherein the vector is a high copy number vector selected from the group consisting of: pHT304, pHT315, and pHT370; or wherein the vector is a low copy number vector selected from the group consisting of: pHT73, and pBMB299.

10. A vector comprising the expression system of claim 4.

11. A recombinant strain of genus Bacillus comprising the expression system of claim 4.

12. The recombinant strain of genus Bacillus of claim 11, wherein the recombinant strain comprises a strain of Bacillus thuringiensis.

13. The recombinant strain of genus Bacillus of claim 11, wherein the recombinant strain comprises a non-sporulating strain.

14. The recombinant strain of genus Bacillus of claim 11, wherein the recombinant strain expresses Vip3A and at least one Cry toxin.

15. A biopesticide comprising the recombinant strain of genus Bacillus of claim 14.
